SEQUENCE LISTING (26 sequences)

SEQ ID NO: 1 - protein, 12 aa, Artificial Sequence
  Inhibitory peptide
SWELYYPLRANL

SEQ ID NO: 2 - protein, 7 aa, Artificial Sequence
  Inhibitory peptide
SWELYYP

SEQ ID NO: 3 - protein, 5 aa, Homo sapiens
CHAVC

SEQ ID NO: 4 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 5 - protein, 268 aa, Homo sapiens
MPRSFLVKKHFNASKKPNYSELDTHTVIISPYLYESYSMPVIPQPEILSSGAYSPITVWTTAAPFHAQLPNGLSPLSGYSSSLGRVSPPPPSDTSSKDHSGSESPISDEEERLQSKLSDPHAIEAEKFQCNLCNKTYSTFSGLAKHKQLHCDAQSRKSFSCKYCDKEYVSLGALKMHIRTHTLPCVCKICGKAFSRPWLLQGHIRTHTGEKPFSCPHCNRAFADRSNLRAHLQTHSDVKKYQCKNCSKTFSRMSLLHKHEESGCCVAH

SEQ ID NO: 6 - protein, 264 aa, Homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 7 - protein, 264 aa, Mus musculus
MPRSFLVRKPSDPRRKPNYSELQDACVEFTFQQPYDQAHLLAAIPPPEVLNPAASLPTLIWDSLLVPQVRPVAWATLPLRESPKAVELTSLSDEDSGKSSQPPSPPSPAPSSFSSTSASSLEAEAFIAFPGLGQLPKQLARLSVAKDPQSRKIFNCKYCNKEYLSLGALKMHIRSHTLPCVCTTCGKAFSRPWLLQGHVRTHTGEKPFSCSHCNRAFADRSNLRAHLQTHSDVKRYQCQACARTFSRMSLLHKHQESGCSGGPR

SEQ ID NO: 8 - protein, 1214 aa, Homo sapiens
MKQPIMADGPRCKRRKQANPRRKNVVNYDNVVDTGSETDEEDKLHIAEDDGIANPLDQETSPASVPNHESSPHVSQALLPREEEEDEIREGGVEHPWHNNEILQASVDGPEEMKEDYDTMGPEATIQTAINNGTVKNANCTSDFEEYFAKRKLEERDGHAVSIEEYLQRSDTAIIYPEAPEELSRLGTPEANGQEENDLPPGTPDAFAQLLTCPYCDRGYKRLTSLKEHIKYRHEKNEENFSCPLCSYTFAYRTQLERHMVTHKPGTDQHQMLTQGAGNRKFKCTECGKAFKYKHHLKEHLRIHSGEKPYECPNCKKRFSHSGSYSSHISSKKCIGLISVNGRMRNNIKTGSSPNSVSSSPTNSAITQLRNKLENGKPLSMSEQTGLLKIKTEPLDFNDYKVLMATHGFSGTSPFMNGGLGATSPLGVHPSAQSPMQHLGVGMEAPLLGFPTMNSNLSEVQKVLQIVDNTVSRQKMDCKAEEISKLKGYHMKDPCSQPEEQGVTSPNIPPVGLPVVSHNGATKSIIDYTLEKVNEAKACLQSLTTDSRRQISNIKKEKLRTLIDLVTDDKMIENHNISTPFSCQFCKESFPGPIPLHQHERYLCKMNEEIKAVLQPHENIVPNKAGVFVDNKALLLSSVLSEKGMTSPINPYKDHMSVLKAYYAMNMEPNSDELLKISIAVGLPQEFVKEWFEQRKVYQYSNSRSPSLERSSKPLAPNSNPPTKDSLLPRSPVKPMDSITSPSIAELHNSVTNCDPPLRLTKPSHFTNIKPVEKLDHSRSNTPSPLNLSSTSSKNSHSSSYTPNSFSSEELQAEPLDLSLPKQMKEPKSIIATKNKTKASSISLDHNSVSSSSENSDEPLNLTFIKKEFSNSNNLDNKSTNPVFSMNPFSAKPLYTALPPQSAFPPATFMPPVQTSIPGLRPYPGLDQMSFLPHMAYTYPTGAATFADMQQRRKYQRKQGFQGELLDGAQDYMSGLDDMTDSDSCLSRKKIKKTESGMYACDLCDKTFQKSSSLLRHKYEHTGKRPHQCQICKKAFKHKHHLIEHSRLHSGEKPYQCDKCGKRFSHSGSYSQHMNHRYSYCKREAEEREAAEREAREKGHLEPTELLMNRAYLQSITPQGYSDSEERESMPRDGESEKEHEKEGEDGYGKLGRQDGDEEFEEEEEESENKSMDTDPETIRDEEETGDHSMDDSSEDGKMETKSDHEEDNMEDGM

SEQ ID NO: 9 - protein, 1215 aa, Mus musculus
MKQPIMADGPRCKRRKQANPRRKNVVNYDNVVDAGSETDEEDKLHIAEDDSLANPLDQDTSPASMPNHESSPHMSQGLLPREEEEEELRESVVEHSWHSGEILQASVAGPEEMKEDYDAMGPEATIQTTINNGTVKNANCTSDFEEYFAKRKLEERDGHAVSIEEYLQRSDTAIIYPEAPEELSRLGTPEANGQEENDLPPGTPDAFAQLLTCPYCDRGYKRLTSLKEHIKYRHEKNEENFSCPLCSYTFAYRTQLERHMVTHKPGTDQHQMLTQGAGNRKFKCTECGKAFKYKHHLKEHLRIHSGEKPYECPNCKKRFSHSGSYSSHISSKKCIGLISVNGRMRNNIKTGSSPNSVSSSPTNSAITQLRNKLENGKPLSMSEQTGLLKIKTEPLDFNDYKVLMATHGFSGSSPFMNGGLGATSPLGVHPSAQSPMQHLGVGMEAPLLGFPTMNSNLSEVQKVLQIVDNTVSRQKMDCKTEDISKLKGYHMKDPCSQPEEQGVTSPNIPPVGLPVVSHNGATKSIIDYTLEKVNEAKACLQSLTTDSRRQISNIKKEKLRTLIDLVTDDKMIENHSISTPFSCQFCKESFPGPIPLHQHERYLCKMNEEIKAVLQPHENIVPNKAGVFVDNKALLLSSVLSEKGLTSPINPYKDHMSVLKAYYAMNMEPNSDELLKISIAVGLPQEFVKEWFEQRKVYQYSNSRSPSLERTSKPLAPNSNPTTKDSLLPRSPVKPMDSITSPSIAELHNSVTSCDPPLRLTKSSHFTNIKAVDKLDHSRSNTPSPLNLSSTSSKNSHSSSYTPNSFSSEELQAEPLDLSLPKQMREPKGIIATKNKTKATSINLDHNSVSSSSENSDEPLNLTFIKKEFSNSNNLDNKSNNPVFGMNPFSAKPLYTPLPPQSAFPPATFMPPVQTSIPGLRPYPGLDQMSFLPHMAYTYPTGAATFADMQQRRKYQRKQGFQGDLLDGAQDYMSGLDDMTDSDSCLSRKKIKKTESGMYACDLCDKTFQKSSSLLRHKYEHTGKRPHQCQICKKAFKHKHHLIEHSRLHSGEKPYQCDKCGKRFSHSGSYSQHMNHRYSYCKREAEEREAAEREAREKGHLGPTELLMNRAYLQSITPQGYSDSEERESMPRDGESEKEHEKEGEEGYGKLRRRDGDEEEEEEEEESENKSMDTDPETIRDEEETGDHSMDDSSEDGKMETKSDHEEDNMEDGMG

SEQ ID NO: 10 - protein, 654 aa, Homo sapiens
MNQPQRMAPVGTDKELSDLLDFSMMFPLPVTNGKGRPASLAGAQFGGSGLEDRPSSGSWGSGDQSSSSFDPSRTFSEGTHFTESHSSLSSSTFLGPGLGGKSGERGAYASFGRDAGVGGLTQAGFLSGELALNSPGPLSPSGMKGTSQYYPSYSGSSRRRAADGSLDTQPKKVRKVPPGLPSSVYPPSSGEDYGRDATAYPSAKTPSSTYPAPFYVADGSLHPSAELWSPPGQAGFGPMLGGGSSPLPLPPGSGPVGSSGSSSTFGGLHQHERMGYQLHGAEVNGGLPSASSFSSAPGATYGGVSSHTPPVSGADSLLGSRGTTAGSSGDALGKALASIYSPDHSSNNFSSSPSTPVGSPQGLAGTSQWPRAGAPGALSPSYDGGLHGLQSKIEDHLDEAIHVLRSHAVGTAGDMHTLLPGHGALASGFTGPMSLGGRHAGLVGGSHPEDGLAGSTSLMHNHAALPSQPGTLPDLSRPPDSYSGLGRAGATAAASEIKREEKEDEENTSAADHSEEEKKELKAPRARTSPDEDEDDLLPPEQKAEREKERRVANNARERLRVRDINEAFKELGRMCQLHLNSEKPQTKLLILHQAVSVILNLEQQVRERNLNPKAACLKRREEEKVSGVVGDPQMVLSAPHPGLSEAHNPAGHM

SEQ ID NO: 11 - DNA, 4396 bp, Homo sapiens
gcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcgctgggtgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtatgcctccgtgggacgagccacccgctttcagccctgtgctctggccccagaagccggactcgagaccccgggcttcatccacatccacacctcacacacctgttgtcagcatcgagccaacaccaacctgacaaggttcggagtgatgggggcggccaaggtgacactgggtccaggagctccctggggccctggcctaccactcactggcctcgctccccctgtccccgaatctcagccaccgtgtcactctgtgacctgtcccatggatcctgaaactgcatcttggccctgttgcctgggctgacaggagcattttttttttttccagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaaggtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagccccccccgtccccccccccggaagccactgcacacagacagacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggccccagcggtaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactccttgccccagggacccaggctcatgctctgaactctggctgagaggagtctgctcaggagccagcacaggacaccccccaccccaccccaccatgtccccattacaccagagggccatcgtgacgtagacaggatgccaggggcctgaccagcctccccaatgctggggagcatccctggcctggggccacacctgctgccctccctctgtgtggtccaagggcaagagtggctggagccgggggactgtgctggtctgagccccacgaaggccttgggctgtggctccgaccctgctgcagaaccagcagggtgtcccctcgggcccatctgtgtcccatgtcccagcacccaggcctctctccaggtctccttttctggtcttttgccatgagggtaaccagctcttcccagctggctgggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctgcgccctgggagtttgatctcttaagggaacttgcctctccctcttgttttgctcctgccctgcccctaggtctgggtggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaattttttttaaagaaaataattgtatacgtgtcaacacagctggctggatgattgggactttaaaacgaccctctttcaggtggattcagagacctgtcctgtatataacagcactgtagcaataaacgtgacattttataaag

SEQ ID NO: 12 - protein, 648 aa, Mus musculus
MMNQSQRMAPVGSDKELSDLLDFSMMFPLPVANGKSRPASLGGTQFAGSGLEDRPSSGSWGSSDQNSSSFDPSRTYSEGAHFSDSHSSLPPSTFLGAGLGGKGSERNAYATFGRDTSVGTLSQAGFLPGELSLSSPGPLSPSGIKSSSQYYPSFPSNPRRRAADGGLDTQPKKVRKVPPGLPSSVYPPSSGDSYSRDAAAYPSAKTPSSAYPSPFYVADGSLHPSAELWSTPSQVGFGPMLGDGSSPLPLAPGSSSVGSGTFGGLQQQDRMGYQLHGSEVNGSLPAVSSFSAAPGTYSGTSGHTPPVSGAAAESLLGTRGTTASSSGDALGKALASIYSPDHSSNNFSPSPSTPVGSPQGLPGTSQWPRAGAPSALSPNYDAGLHGLSKMEDRLDEAIHVLRSHAVGTASDLHGLLPGHGALTTSFTGPMSLGGRHAGLVGGSHPEEGLTSGASLLHNHASLPSQPSSLPDLSQRPPDSYSGLGRAGTTAGASEIKREEKEDEEIASVADAEEDKKDLKVPRTRTSSTDEVLSLEEKDLRDRERRMANNARERVRVRDINEAFRELGRMCQLHLKSDKAQTKLLILQQAVQVILGLEQQVRERNLNPKAACLKRREEEKVSGVVGDPQLPLSAAHPGLGEAHNPAGHL

SEQ ID NO: 13 - DNA, 2898 bp, Mus musculus
gcgccggcggctgcgggcgtagcgggccaccgcgggccaccgccgcgcgccgccgcctctgctacagtcccttcccgcggggcctgctctgagagaagctcgagagagaccaggcgacgcgaacgcgagtggggaggaggaaggacgcgcgaccccgagccctgcgcgctcccgccgcccacgcgcgaccctcggggacgcgcccgccacccttttgtccccggggtccccgagggcggtgggcagcagggagccccggtgcacccggtgcatgcccccgcccagcagggctgtctctagacctgggggacgcaccccagttccaacacctgctgtcctgggtggatgatgaaccagtctcagagaatggcacccgtgggctctgacaaggaactgagtgacctcctggacttcagcatgatgttcccgctacctgtggccaatgggaagagccggcccgcctccctcgggggaacccagtttgcaggctcaggactggaggaccgacccagctcaggctcctggggcagcagtgaccagaacagttcttcctttgaccctagccggacatacagcgaaggtgcccacttcagtgactcccacagcagcctgccgccttccacgttcctaggagctgggcttggaggcaagggcagtgagcggaatgcctatgccacctttgggagagacaccagtgttggcaccttgagtcaggctggcttcctgccaggtgagctgagcctcagcagtcccgggccactgtccccatcgggcatcaagagcagctcccagtattacccctcattccccagcaaccctcgtcggagagctgcagatggtggcctggatactcagccgaagaaggtccggaaggttccgcctggtctcccttcctcggtgtatccgcccagctcaggtgacagctacagcagggatgctgcagcctacccctccgccaagacccccagcagcgcttacccctcccccttctacgtggcagatggcagcctgcacccatcagctgagctctggagtacgcctagccaggtgggctttgggcccatgctaggtgacggctcttcccctctgccccttgcaccgggcagcagctccgtgggcagtggtacctttgggggcctccagcagcaggatcgcatgggctaccagctgcatggatctgaggttaatggctcgctcccagctgtatccagcttttcggctgcccctggcacttacagtgggacttccggccacacgccccctgtgagtggggccgcagctgaaagcctcctaggcacccgagggactacagccagcagctcaggggatgcccttgggaaggcactggcctcgatctactccccggatcactccagcaataatttctcacctagcccctcaacgcctgtgggttcaccccagggcctgccagggacatcacagtggccccgggcaggagcgcccagtgccttatcccccaactacgatgcaggtctccatggcctgagcaagatggaggaccgcttggacgaggccatccatgtcctgcgaagccacgctgttggcaccgctagcgatctccatgggcttttgcctggccatggcgcactgaccacgagcttcaccggccccatgtcactgggcgggcggcatgccggcctggtcgggggaagccatcctgaggagggcctcacaagtggggccagtcttttgcataaccatgccagcctccccagccagcccagttccctccctgacctctcacagagacctcccgactcctatagtggactcgggagggcaggcacaacagcgggtgccagcgagatcaagcgggaggagaaagaggatgaggaaatcgcatcagtagccgacgccgaagaggacaagaaggacctgaaggtcccacgcacgcgcaccagcagtacagatgaggtgctgtccctggaggagaaggacctgagggaccgggagaggcgtatggccaataacgctcgggagcgggtgcgcgtgcgggacattaacgaggccttccgggagctgggccgcatgtgccagctgcacctcaagtcggataaggcgcagaccaagctgctcatcctgcagcaggcggtgcaggtcatcctgggcctggagcagcaggtgcgagaacgcaacctgaaccccaaagcagcctgcttgaagcggagggaggaggagaaggtgtctggcgtggtcggggacccacagctgcccctgtcagccgcccacccgggcctgggtgaggcccacaacccagccgggcacctgtgagccgtcacagcttcttcgttggaccagggaccaccatatctctgcccggggtgcatcaggacggttctggatgagacaggtctccatcgaagcatgagcagagagagggctctggggacacttcagggcctggggagggtggcactgaacagctccctgcttggccccagtgaccaagcagaaaagttccttcctctcggttaaccagaactggaaacaaagcagcatgctcccttttcaaaaaggaaagaaagatgccttaactatgtaagacggaagagtcggaccgtgccctggcagggcggcctgggactggcttctacttcagagccaccagcacatcgtgcctaagcatttttcgtttttttaaaggagaataaaggaacattagttttcagattttttttttaaatgtagacaaaagttagcaagaacgaggccttccgtgtcttttttttttcccttagcttttttttccgtatgttttgtaagcaacaaatttttgtataaaagtctcatgtctgtttctgtttctagaaaaaaaaaaaaaaaaaaaaaaaaaaatatttaaaaaaaaaaaaaaaaaaaaaaaaaaaaaaaaaaaaa

SEQ ID NO: 14 - DNA, 76 bp, Artificial
  Human siRNA insert 1
ggatcccgtatttacgacctttcttggcattgatatccgtgccaagaaaggtcgtaaatattttttccaaaagctt

SEQ ID NO: 15 - DNA, 76 bp, Artificial
  Human siRNA insert 2
ggatcccgtttcttgagccataaatgctcttgatatccggagcatttatggctcaagaaattttttccaaaagctt

SEQ ID NO: 16 - DNA, 76 bp, Artificial
  Human siRNA insert 3
ggatcccgttagtgagtcagcaaattgatttgatatccgatcaatttgctgactcactaattttttccaaaagctt

SEQ ID NO: 17 - DNA, 76 bp, Artificial
  Human siRNA insert 4
ggatcccgtgtgagccatgagccactgagttgatatccgctcagtggctcatggctcacattttttccaaaagctt

SEQ ID NO: 18 - DNA, 76 bp, Artificial
  Human siRNA insert 5
ggatcccgcatagtcaacaaccaggcaggttgatatccgcctgcctggttgttgactatgttttttccaaaagctt

SEQ ID NO: 19 - DNA, 76 bp, Artificial
  Murine siRNA insert 1
ggatcccgttgttctggttatccgcgagcttgatatccggctcgcggataaccagaacaattttttccaaaagctt

SEQ ID NO: 20 - DNA, 76 bp, Artificial
  Murine siRNA insert 2
ggatcccgtctgtgacgacaacgaactgcttgatatccggcagttcgttgtcgtcacagattttttccaaaagctt

SEQ ID NO: 21 - DNA, 76 bp, Artificial
  Murine siRNA insert 3
ggatcccgtagatgccgcttcactgtgatttgatatccgatcacagtgaagcggcatctattttttccaaaagctt

SEQ ID NO: 22 - protein, 269 aa, Mus musculus
MPRSFLVKKHFNASKKPNYSELDTHTVIISPYLYESYPIPVIPKPEILTSGAYSPITVWTSSAAPLHSPLPSGLSPLTGYSSSLGRVSPPPSSDTSSKDHSGSESPISDEEERLQPKLSDPHAIEAEKFQCNLCNKTYSTFSGLAKHKQLHCDAQSRKSFSCKYCDKEYVSLGALKMHIRTHTLPCVCKICGKAFSRPWLLQGHIRTHTGEKPFSCPHCNRAFADRSNLRAHLQTHSDVKKYQCKNCSKTFSRMSLLHKHEESGCCVAH

SEQ ID NO: 23 - DNA, 4828 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgccggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgcctttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaattttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccataaaaaaaaaaaaa

SEQ ID NO: 24 - protein, 884 aa, Mus musculus
MGARCRSFSALLLLLQVSSWLCQELEPESCSPGFSSEVYTFPVPERHLERGHVLGRVRFEGCTGRPRTAFFSEDSRFKVATDGTITVKRHLKLHKLETSFLVRARDSSHRELSTKVTLKSMGHHHHRHHHRDPASESNPELLMFPSVYPGLRRQKRDWVIPPISCPENEKGEFPKNLVQIKSNRDKETKVFYSITGQGADKPPVGVFIIERETGWLKVTQPLDREAIAKYILYSHAVSSNGEAVEDPMEIVITVTDQNDNRPEFTQPVFEGFVAEGAVPGTSVMKVSATDADDDVNTYNAAIAYTIVSQDPELPHKNMFTVNRDTGVISVLTSGLDRESYPTYTLVVQAADLQGEGLSTTAKAVITVKDINDNAPVFNPSTYQGQVPENEVNARIATLKVTDDDAPNTPAWKAVYTVVNDPDQQFVVVTDPTTNDGILKTAKGLDFEAKQQYILHVRVENEEPFEGSLVPSTATVTVDVVDVNEAPIFMPAERRVEVPEDFGVGQEITSYTAREPDTFMDQKITYRIWRDTANWLEINPETGAIFTRAEMDREDAEHVKNSTYVALIIATDDGSPIATGTGTLLLVLLDVNDNAPIPEPRNMQFCQRNPQPHIITILDPDLPPNTSPFTAELTHGASVNWTIEYNDAAQESLILQPRKDLEIGEYKIHLKLADNQNKDQVTTLDVHVCDCEGTVNNCMKAGIVAAGLQVPAILGILGGILALLILILLLLLFLRRRTVVKEPLLPPDDDTRDNVYYYDEEGGGEEDQDFDLSQLHRGLDARPEVTRNDVAPTLMSVPQYRPRPANPDEIGNFIDENLKAADSDPTAPPYDSLLVFDYEGSGSEAASLSSLNSSESDQDQDYDYLNEWGNRFKKLADMYGGGEDD

SEQ ID NO: 25 - DNA, 3314 bp, Mus musculus
agccgcggcgcactactgagttcccaagaacttctgctagactcctgcccggcctaacccggccctgcccgaccgcacccgagctcagtgtttgctcggcgtctgccgggtccgccatgggagcccggtgccgcagcttttccgcgctcctgctcctgctgcaggtctcctcatggctttgccaggagctggagcctgagtcctgcagtcccggcttcagttccgaggtctacaccttcccggtgccggagaggcacctggagagaggccatgtcctgggcagagtgagatttgaaggatgcaccggccggccaaggacagccttcttttcggaagactcccgattcaaagtggcgacagacggcaccatcacagtgaagcggcatctaaagctccacaagctggagaccagtttcctcgtccgcgcccgggactccagtcatagggagctgtctaccaaagtgacgctgaagtccatggggcaccaccatcaccggcaccaccaccgcgaccctgcctctgaatccaacccagagctgctcatgtttcccagcgtgtacccaggtctcagaagacagaaacgagactgggtcatccctcccatcagctgccccgaaaatgaaaagggtgaattcccaaagaacctggttcagatcaaatccaacagggacaaagaaacaaaggttttctacagcatcaccggccaaggagctgacaaaccccccgttggcgttttcatcattgagagggagacaggctggctgaaagtgacacagcctctggatagagaagccattgccaagtacatcctctattctcatgccgtgtcatcaaatggggaagcggtggaggatcccatggagatagtgatcacagtgacagatcagaatgacaacaggccagagtttacccaggaggtgtttgagggatccgttgcagaaggcgctgttccaggaacctccgtgatgaaggtctcagccaccgatgcagacgatgacgtcaacacctacaacgctgccatcgcctacaccatcgtcagccaggatcctgagctgcctcacaaaaacatgttcactgtcaatagggacaccggggtcatcagtgtgctcacctctgggctggaccgagagagttaccctacatacactctggtggttcaggctgctgaccttcaaggcgaaggcttgagcacaacagccaaggctgtgatcactgtcaaggatattaatgacaacgctcctgtcttcaacccgagcacgtatcagggtcaagtgcctgagaatgaggtcaatgcccggatcgccacactcaaagtgaccgatgatgatgcccccaacactccggcgtggaaagctgtgtacaccgtagtcaacgatcctgaccagcagttcgttgtcgtcacagaccccacgaccaatgatggcattttgaaaacagccaagggcttggattttgaggccaagcagcaatacatccttcatgtgagagtggagaacgaggaaccctttgaggggtctcttgtcccttccacagccactgtcactgtggacgtggtagacgtgaatgaagcccccatctttatgcctgcggagaggagagtcgaagtgcccgaagactttggtgtgggtcaggaaatcacatcttataccgctcgagagccggacacgttcatggatcagaagatcacgtatcggatttggagggacactgccaactggctggagattaacccagagactggtgccattttcacgcgcgctgagatggacagagaagacgctgagcatgtgaagaacagcacatatgtagctctcatcatcgccacagatgatggttcacccattgccactggcacgggcactcttctcctggtcctgttagacgtcaatgataacgctcccatcccagaacctcgaaacatgcagttctgccagaggaacccacagcctcatatcatcaccatcttggatccagaccttccccccaacacgtccccctttactgctgagctaacccatggggccagcgtcaactggaccattgagtataatgacgcagctcaagaatctctcattttgcaaccaagaaaggacttagagattggcgaatacaaaatccatctcaagctcgcggataaccagaacaaagaccaggtgaccacgttggacgtccatgtgtgtgactgtgaagggacggtcaacaactgcatgaaggcgggaatcgtggcagcaggattgcaagttcctgccatcctcggaatccttggagggatcctcgccctgctgattctgatcctgctgctcctactgtttctacggaggagaacggtggtcaaagagcccctgctgccaccagatgatgatacccgggacaatgtgtattactatgatgaagaaggaggtggagaagaagaccaggactttgatttgagccagctgcacaggggcctggatgcccgaccggaagtgactcgaaatgatgtggctcccaccctcatgagcgtgccccagtatcgtccccgtcctgccaatcctgatgaaattggaaacttcatcgatgaaaacctgaaggcagccgacagcgaccccacggcacccccttacgactctctgttggtgttcgattacgagggcagtggttctgaagccgctagcctgagctcactgaactcctctgagtcggatcaggaccaggactacgattatctgaacgagtggggcaaccgattcaagaagctggcggacatgtacggcggtggcgaggacgactaggggactagcaagtctcccccgtgtggcaccatgggagatgcagaataattatatcagtggtctttcagctccttccctgagtgtgtagaagagagactgatctgagaagtgtgcagattgcatagtggtctcattctccttactggactgtctgtgttaggatggttttcactgattgttgaaatctttttttattttttatttttacagtgctgagatataaactgtgcctttttttgtttgtttgtttctgtttttgttcttttgagcagaacaaaaaaaagggaccactatgcatgctgcacacgtctcagattcttaggtacacacctgattcttaggtgcatgccatagtgggatatgttgctttgatcagaacctgcagggaggttttcgggcaccacttaagtttcttggcgtttctttcaaaccgttctctaagatgcatttttatgaattttattaaagagttttgttaaaaaaaaaaaaaaaaaaaaaaaaaaaaaaaaaaaaaaaaaaaaaaaaaaaaaaaaaaaaaaaaaa

SEQ ID NO: 26 - protein, 8 aa, Artificial Sequence
  Inhibitory peptide
SWELYYPL

(19) United States
(12) Patent Application Publication
(10) Pub. No.: US 2015/0291933 A1
(43) Pub. Date: Oct. 15, 2015

(54) CELL DIFFERENTIATION

(71) Applicant: THE UNIVERSITY OF MANCHESTER, Manchester (GB)

(72) Inventors: Lisa Mohamet, Manchester (GB); Christopher Ward, Manchester (GB)

(21) Appl. No.: 14/441,012

(22) PCT Filed: Nov. 7, 2013
(86) PCT No.: PCT/GB2013/052924

§ 371 Date: May 6, 2015

(30) Foreign Application Priority Data
Nov. 7, 2012 (GB) 1220058.0

(51) Int. Cl. C12N 5/0793; C12N 5/079

(52) U.S. Cl. CPC C12N 5/0619; C12N 5/0622; C12N 2501/58; C12N 2506/02; C12N 2506/45

(57) ABSTRACT

Provided is a method of producing neural precursor cells, in which an inhibitor of E-cadherin activity is provided to a population of the cells having neural potential, cell stress is induced among the population of cells; and the surviving cells are cultured until neural precursor cells are produced. Also provided is a method of adapting a cell in vitro for therapeutic use, in which an inhibitor of E-cadherin activity is provided to a population of cells having neural potential, cell stress is induced among the population of cells, and the surviving cells are cultured until neural precursor cells are produced. This method may optionally additionally involve culturing the neural precursor cells until neural cells are produced and formulating the neural precursor cells or neural cells in a composition suitable for administration to a patient. The invention also provides cells produced by these methods. The methods may be practiced on stem cells, particularly iPSCs. The cells and methods have utility in applications including stratified medicine.

The present invention relates to methods of producing neural precursor cells and/or neural cells. The invention also relates to cells produced by such methods, and kits and cell culture media suitable for use in methods of the invention. The methods, cells, kits and cell culture media have a range of applications, including in the development and implementation of stratified medicines.

### INTRODUCTION

Reproducible, cost-effective and scalable production of specific cell types can impact on a wide range of applications ranging from reliable in vitro assays for drug efficiency and toxicity testing, to cellular therapy. The ability to produce neural precursor cells and/or neural cells in this manner may open the possibility of such assays, testing, and therapy being made available in respect of conditions that adversely impact the nervous system, including diseases, such as Alzheimer's or Parkinson's disease, and nervous system injuries.

Current protocols in which desired cell types are produced by differentiation of pluripotent stem cells make use of complex and expensive growth factor cocktails. The inventors have developed a novel process that directs differentiation of ES cells to neural lineages in the absence of exogenous growth factors, providing a scalable, highly efficient, cost-effective and reproducible method.

The cadherins are a family of integral membrane proteins which are involved in calcium-dependent cell adhesion. E-cadherin is so called because of its association with the epithelium. Classical cadherins comprise an extracellular domain of approximately 600 amino acid residues, a transmembrane domain, and an intracellular domain of 150 amino acid residues. The extracellular domain comprises four repeated sequences that are believed to be associated with calcium ion binding. The gene encoding E-cadherin is known as cdh1.

The amino acid sequence of human E-cadherin is set out in SEQ ID NO. 4, while the sequence of DNA encoding this protein is set out in SEQ ID NO. 23. The amino acid sequence of mouse E-cadherin is set out in SEQ ID NO. 24, and the sequence of DNA encoding this protein is set out in SEQ ID NO. 25.

There is a need for methods, kits, and cell culture media that can be used for the production of neural precursor cells and/or neural cells in the absence of exogenous growth factors. Such methods, kits, or culture media may be of benefit in providing routes of production that are scalable, and/or efficient, and/or cost-effective, and/or reproducible as compared to the methods of the prior art.

It is an aim of at least some aspects of the present invention to provide methods of producing neural precursor cells that are purer than the populations of such cells that can be produced using prior art methods. It is an aim of at least some aspects of the present invention to provide methods of producing neural precursor cells that have a greater degree of reproducibility than do prior art methods. It is an aim of at least some aspects of the present invention to provide methods of producing neural precursor cells that are simpler than prior art methods. It is an aim of at least some aspects of the present invention to provide methods of producing neural precursor cells that are more cost effective than prior art methods.

In a first aspect of the invention there is provided a method of producing neural precursor cells, the method comprising:
providing an inhibitor of E-cadherin activity to a population of the cells having neural potential;
inducing physiological stress among the population of cells; and
   culturing the surviving cells until neural precursor cells are produced.

The stress induced in the cells may be sufficient to cause cell death among the population.

The inventors have surprisingly found that by inducing stress or cell death among populations of cells in which E-cadherin activity is inhibited, and then expanding the numbers of surviving cells in culture, they are able to produce cell populations comprising high proportions of neural precursor cells. It will be appreciated that intentionally stressing a population of cells that are being cultured with a view to obtaining cells of a desired type, even to the point of inducing cell death among the cultured cells, is counter-intuitive. Inducing stress or cell death in this manner would be expected to undesirably reduce total cell numbers, without any expectation that this would have a beneficial effect upon the nature of the cells remaining.

The Experimental Results described in more detail elsewhere in the specification, describe methods of the invention producing populations of cells in which neural precursor cells and/or neural cells account for 95% or more of total cell numbers. These proportions are significantly higher than those produced using comparable control techniques.

The methods of the invention are able to give rise to populations of neural precursor cells and/or neural cells that have high purity compared to those produced by alternative methods. In suitable embodiments the methods of the invention may give rise to populations comprising at least 70% neural precursor cells, at least 75% neural precursor cells, at least 80% neural precursor cells, or more. By way of example, in suitable embodiments the methods of the invention may give rise to populations comprising at least 85% neural precursor cells, at least 90% neural precursor cells, or more. In certain embodiments, the methods of the invention may give rise to populations comprising at least 91% neural precursor cells, at least 92% neural precursor cells, at least 93% neural precursor cells, at least 94% neural precursor cells, at least 95% neural precursor cells, at least 96% neural precursor cells, at least 97% neural precursor cells, at least 98% neural precursor cells, or at least 99% neural precursor cells. In certain embodiments, the methods of the invention may give rise to substantially pure populations of neural precursor cells.

These purities of such populations are considerably greater than those that may be achieved using comparator methods, or methods of the prior art. By way of example, comparator methods in which the same physiological stress is applied to cells, but the inhibitor of E-cadherin activity is omitted, yield populations comprising a maximum of 70% neural precursor cells. The most effective methods described in the prior art yield populations comprising a maximum of 90% neural precursor cells.

The methods of the invention also offer a number of other advantages in addition to the improved purity of cell populations that they are able to yield. The methods of the invention are simpler than many methods currently available. Many prior art methods make us of protocols that involve three or four separate steps, including suspension culture. In contrast, the methods of the invention may be practiced in a single step protocol, making use of adherent culture, by simple medium supplementation and embodiments in which removal of exogenous signals provides physiological stress.

Furthermore, the methods of the invention are highly reproducible, which provides a notable benefit offered over prior art methods that predominantly rely on the use of exogenous growth factors to control differentiation. Since such growth factors frequently exhibit large variability between batches there can be significant variation in the cell populations that they give rise to, even when other variables are appropriately controlled for.

A further advantage offered by the methods of the invention is that they may be put into practice more cheaply than many prior art techniques. For example, the methods of the invention can be practiced more cheaply than techniques that require the use of expensive exogenous growth factors.

Without wishing to be bound by any hypothesis, the inventors believe that, while the methods of the invention are effective in cells derived from many different types of animal, they may bring about their actions in different animals by different means.

In the case of production of neural precursor cells and/or neural cells from human cells and cell cultures, the inventors believe that the induction of physiological stress induces differentiation of the cells that survive, but the presence of the E-cadherin inhibitor retards differentiation along the majority of cell lineages, though it surprisingly does not retard differentiation into neural precursor cells, thus causing these cells to be produced.

In contrast, in the case of murine cells, the inventors believe that E-cadherin inhibition protects a sub-population of cells that will then give rise to neural precursor cells. By inducing cell death the cells other than those of this sub-population are substantially removed, thus yielding neural precursor cell populations of high purity. The generation of neural precursor cells in murine cell populations in this manner occurs in particular when the methods of the invention are applied to cells grown in suspension culture.

In certain embodiments, the methods of the invention may optionally comprise a further step of culturing the neural precursor cells until neural cells are produced. Thus the invention may also provide methods of producing neural cells. Such embodiments may make use of culture conditions that favour differentiation of neural precursor cells into neural cells, and such conditions described in greater detail elsewhere in the present specification.

In alternative embodiments, the methods of the invention may optionally comprise a further step of culturing the neural precursor cells until glial cells, such as oligodendrocyte or astrocytes are produced. Thus the invention may also provide methods of producing glial cells (such as oligodendrocytes or astrocytes). Embodiments of this sort may make use of culture conditions that favour differentiation of neural precursor cells into glial cells, and suitable examples of such conditions, which may be used to favour differentiation into oligodendrocyte or astrocyte cells, are described in greater detail elsewhere in the present specification.

As set out in more detail below, stem cells are an example of cells having neural potential that may be used in the methods of the invention.

Without precluding other alternatives, the inhibitor of E-cadherin activity may be an exogenous inhibitor of E-cadherin activity. Suitably, for example in embodiments where the inhibitor of E-cadherin activity is an exogenous inhibitor, the inhibitor may be provided in a culture medium. More details regarding suitable inhibitors of E-cadherin activity are provided elsewhere in the specification.

When practicing the methods of the invention, the cells should be subject to inhibition of E-cadherin activity at the time when physiological stress is induced among cells. This may be achieved, for example, by provision of an inhibitor of E-cadherin activity prior to, or concurrently with, the induction of stress.

It will be appreciated that most methods for inducing physiological stress, and potentially cell death, in a cell population will not achieve instantaneous results. Accordingly the methods of the invention may remain effective if a suitable means of inducing physiological stress is provided to the population of cells at the same time as the provision of the inhibitor of E-cadherin activity. Such embodiments may still prove effective on the proviso that the inhibitor will be able to exert at least some inhibition prior to physiological stress occurring. Alternatively, stress may be induced in the population of cells following provision of the inhibitor of E-cadherin activity.

In the case of methods practiced in respect of human cells, the inventors believe that it is highly desirable to inhibit E-cadherin activity during at least the initial five, six, or preferably seven days after physiological stress is induced among cells.

Embodiments utilising induction of cell death may involve inducing the death of up to 85% of the cultured cells. It will be appreciated that the proportion of cells dying may increase over time during the practice of a method of the invention. Merely by way of example, on the first day of a method of the invention death of approximately 2% of the cell population may be induced. By the third day of a method of the invention, death of approximately 23% of the cell population may be induced. By the sixth day of a method of the invention, death of approximately 69% of the cell population may be induced. By the ninth day of a method of the invention, death of approximately 79% of the cell population may be induced. By the twelfth day of a method of the invention death of approximately 81% of the cell population may be induced. By the fifteenth day of a method of the invention death of approximately 85% of the cell population may be induced. The above values may be particularly appropriate in respect of methods of the invention practiced in respect of murine cells.

Physiological stress, and optionally cell death, may be induced in the population of cultured cells by many suitable different means. For example, physiological stress, and optionally cell death, may be induced among the population of cells by withdrawal of an agent that is beneficial to cultured cells, such as withdrawal of beneficial media supplements. For example, physiological stress, and optionally cell death, may be induced by withdrawal of serum from the medium provided to the cell population that have previously been maintained in cell culture medium containing serum or a serum replacement composition.

Another approach which may be used to augment physiological stress that may be induced in a population of cells is to maintain the cells at low density at the time that the stress is induced. This may serve to inhibit cell to cell contact, and remove conditions that would help the cells to maintain pluripotency. For example, cells may be maintained at a density corresponding to less than 80% confluence, less than 70% confluence or less than 60% confluence at the time that the physiological stress is induced. In suitable embodiments the cells may be at 50% confluence, or less, at the time that the physiological stress is induced.

Alternative methods by which physiological stress may be induced include increasing the temperature to which the population of cells is exposed, increasing or decreasing pH of the medium in which the population of cells is grown, providing a cytotoxic agent to the population of cells.

In embodiments where physiological stress is induced by withdrawal of an agent that is beneficial to the cultured cells this withdrawal may be continued as long as is necessary to induce the requisite physiological stress. In embodiments in which physiological stress is induced by withdrawal of serum from the culture medium the inventors have found that such withdrawal may be continued indefinitely.

In the case of the addition of a stimulus to induce physiological stress, such as a cytotoxic agent, the stimulus may be provided transiently. The stimulus should be provided for sufficient time, and in a sufficient amount, to induce the required extent of physiological stress.

In suitable embodiments the methods of the invention are carried out in vitro. Suitable in vitro methods may involve culturing the cells before and after the provision of the inhibitor of E-cadherin activity. Suitable embodiments may make use of adherent or non-adherent culture methods.

In a second aspect the invention provides a method of adapting a cell in vitro for therapeutic use, the method comprising:
providing an inhibitor of E-cadherin activity to a population of the cells having neural potential;
inducing physiological stress among the population of cells;
culturing the surviving cells until neural precursor cells are produced;
optionally culturing the neural precursor cells until neural cells are produced; and
formulating the neural precursor cells or neural cells in a composition suitable for administration to a patient.

Except for where the context requires otherwise, the various criteria set out in respect of the methods in accordance with the first aspect of the invention may also be applicable to methods in accordance with this second aspect of the invention.

Formulating the neural precursor cells or neural cells may comprise the manufacture of a medicament for the treatment of a condition involving damage to cells of the nervous system. Such a condition may be a disease (such as a neurodegenerative disease) or an injury. Merely by way of example, suitable diseases may include Alzheimer's disease or Parkinson's disease.

The cells for use in methods in accordance with this aspect of the invention may preferably be human cells. In certain embodiments of the methods of this aspect of the invention, the cells may preferably be cells of a patient requiring therapy. The composition may comprise cells from the patient to whom it is for administration.

The cells of a patient requiring treatment also constitute useful materials that may be used in embodiments of the invention other than those relating to direct therapeutic uses of such cells (or their progeny). Merely by way of example, cells of a patient with a disease requiring treatment may be used as a starting material for the production of neural precursor cells, and the response of these neural precursor cells (or their progeny) to potential therapeutic agents investigated. Thus, by way of example, cells of a patient with a disease or disorder of the nervous system may be used to produce neural precursor cells (or their progeny) that exhibit responses or phenotypes characteristic of the disease or disorder in question. The cells may then be exposed to an agent with potential to treat the disease or disorder, and the response of these cells to this potential therapeutic agent assessed. A finding that the potential therapeutic agent is able to alleviate the response or phenotype characteristic of the disease or disorder in question indicates that the same (or similar) agent may be of use in the treatment of the disease or disorder in the patient. By the same token, a finding that a potential therapeutic agent does not alleviate the response indicates that this agent should not be employed in such treatment.

The considerations set out above in respect of therapeutic agents are also applicable to treatment/dosing regimens, and the like.

In a suitable embodiment in which an individual's cells are used in a method of the invention, stem or progenitor cells of the individual may be used directly as the starting material for the method. Alternatively, non-stem cells from the individual may be induced to pluripotency (thus yielding iPSCs) and these iPSCs utilised in the method of the invention.

In a third aspect the invention also provides a kit comprising:
an inhibitor of E-cadherin activity;
a serum-free cell medium; and
serum or a serum-replacement composition.

In a fourth aspect the invention also provides a cell culture medium comprising an inhibitor of E-cadherin activity at a concentration of between approximately 250 μM and approximately 1.3 mM.

In the case of a cell culture medium of the invention for use in the culture of mouse cells, the inhibitor of E-cadherin activity may be provided at a concentration of between 600 μM and 1.3 mM. Suitably the E-cadherin inhibitor may be provided at a concentration of around 1 mM.

In the case of a cell culture medium of the invention for use in the culture of human cells, the inhibitor of E-cadherin activity may be provided at a concentration of between 250 μM and a maximal concentration of 1.3 mM. Suitably the E-cadherin inhibitor may be provided at a concentration of around 500 μM.

In certain embodiments the cell culture medium of the invention is a serum-free medium. In other embodiments the cell culture medium of the invention may comprise serum, or a serum-replacement composition.

It will be recognised that kits or media in accordance with the various embodiments of the invention are well suited to use in the methods of the invention.

Suitable inhibitors of E-cadherin activity for use in the kits or cell culture media of the invention may be selected with reference to the suggestions provided elsewhere in the specification.

### DEFINITIONS

In order that the present invention may be better understood, the following terms are now further defined in the context of the present disclosure. It will be appreciated that, except for where the context requires otherwise, all embodiments considered in the following definitions should be considered suitable for use in all aspects of the invention, irrespective of whether or not the particular combination of the embodiment and aspect is specifically disclosed.

### “Cells Having Neural Potential”

For the purposes of the present disclosure, this term should be taken as encompassing any cells that have the capacity to differentiate and thereby give rise to neural precursor cells. Stem cells are an example of suitable cells having neural potential in the context of the present disclosure.

### “Stem Cells”

As referred to above, stem cells represent a suitable form of cells having neural potential that may be used in the methods of the invention.

In embodiments in which stem cells are utilised, the stem cells may be independently selected from the group consisting of: pluripotent stem cells; multipotent stem cells; totipotent stem cells; adult stem cells; embryonic stem cells; cord blood stem cells; mesenchymal stem cells; epithelial stem cells; adipose stem cells; epi-stem cells; cancer stem cells; and induced pluripotent stem cells (iPSCs). It may be preferred that the stem cells exhibit biological activities (such as pluripotency) associated with “embryonic”, rather than “adult”, stem cell types. Suitable examples of such stem cells exhibiting embryonic characteristics include not only embryonic stem cells, such as embryonic stem cell lines, but also iPSCs. For purposes of patentability, it will be appreciated that in the case of certain embodiments using human stem cells, the human stem cells may be other than human embryonic stem cells.

In certain embodiments of the invention a suitable stem cell line may be one which is produced without requiring the destruction of a human embryo. In a suitable embodiment, a suitable embryonic stem cell line may be one developed by isolation of human embryonic stem cells from early blastocysts. It is known that techniques, such as those in which embryonic stem cells lines are derived from single blastomeres, allow human embryonic stem cells to be isolated and cultured, without harming the embryo from which the cells are taken.

Merely by way of example, the methods of the invention may be practiced using cell lines independently selected from the group consisting of: HUES-7 (Harvard, Melton); H9 (WiCell); MAN-7 (university of Manchester, Kimber); H1 (Wicell); SHEF3 (Sheffield, Moore); iPSCs such as those produced at the University of Manchester (Kapacee); iPS-DF6-9-9T.B--MCB-01 (WiCell); and ENPS cells (D3 (129s2/SvPas parental line—ATCC). The above examples are all human stem cell lines, with the exception of the last cell line referred to, which is murine.

### “Neural Precursor Cells”

In the present context, neural precursor cells may be taken as comprising any cells exhibiting self-renewal and the ability to commit to the neural lineage. Suitable examples of neural precursor cells may include cells capable of giving rise to cell types selected from the group consisting of: neural cells; and neuronal cells; and glial cells, such as oligodendrocyte or astrocytes.

Neural precursor cells may be identified by their profile of expression of certain markers. For example, in suitable embodiments, neural precursor cells may express nestin. Nestin is an intermediate filament expressed primarily in nerve cells. In addition, or as an alternative, to nestin, neural precursor cells produced by the methods of the invention may express one or more markers selected from the group consisting of: SOX-2 and Vimentin.

Expression of suitable markers may be assessed by any suitable technique, including, but not limited to, those selected from the group consisting of: immunolabeling; immunofluorescent microscopy; western blotting; fluorescent activated cell sorting (FACS); fluorescent flow cytometry; polymerase chain reaction (PCR); and reverse transcription PCR (RT-PCR).

In suitable embodiments, neural precursor cells may be distinguished by their morphology, which may be most apparent when grown in adherence culture. Morphological features characteristic of neural precursor cells or neural cells may include the presence of rosette-like structures and a spindle-like morphology. These features are distinguishable from the flattened morphology (referred to as “pavement-like”) of endoderm cells.

Preferably distinguishing morphological features may be used in combination with characteristic markers, for example using immunocytochemistry labelling and microscopy.

Neural precursor cells that have undergone early neural commitment may be identified by expression of a marker selected from the group consisting of: neuron specific β-III tubulin; NEUROD1; and NEUROFILAMENT.

### “Inhibitors of E-Cadherin Activity”

Many different inhibitors of E-cadherin activity are suitable for use in accordance with the present invention. Merely by way of example, suitable inhibitors of E-cadherin activity may be selected from the group consisting of: E-cadherin neutralising antibodies; inhibitors of the E-cadherin HAV domain; inhibitors of tryptophan 2 on the extracellular domain of E-cadherin; and peptides comprising the amino acid sequence CHAVC (SEQ ID NO. 3).

As set out above, E-cadherin neutralising antibodies represent examples of inhibitors of E-cadherin activity suitable for use in accordance with the present invention. Suitable neutralising antibodies are those that, when bound to an epitope present on E-cadherin, and thereby reduce the activity of E-cadherin. For example, the anti-E-cadherin antibody DECMA-I (available from Sigma, Dorset, UK under the catalogue number U3254) may be used as an inhibitor of E-cadherin activity suitable for use in accordance with the invention. Alternatively, a suitable inhibitor of E-cadherin activity may be an antibody other than DECMA-I. One example of a further E-cadherin neutralising antibody that may be used in accordance with the present invention is SHE78-7 (also referred to as SHE78.7), which is commercially available from Zymed Labs, Inc., S. San Francisco, Calif. (Cat. No. 13-5700). DECMA-I antibody was raised against mouse embryonal carcinoma cell line PCC4 Aza RI and SHE78.7 was raised against human placenta, therefore. In the light of this, it will be appreciated that DECMA-I may be more effective at inhibition of E-cadherin activity in mouse (including mouse stem cells such as mouse embryonic stem cells) and SHE78.7 more effective for inhibition of E-cadherin activity in human cells (including human stem cells such as human embryonic stem cells).

In particular, it may be preferred that SHE78.7 be used as an inhibitor of E-cadherin activity when it is wished to inhibit E-cadherin activity associated with human cells. The inventors have found that DECMA-I be used as a preferred inhibitor of E-cadherin activity when it is wished to inhibit E-cadherin activity associated with murine cells.

Antibodies suitable for use as inhibitors of E-cadherin activity in accordance with the present invention include monoclonal activity-neutralizing antibodies and polyclonal activity-neutralizing antibodies, as well as fragments of such antibodies that retain the neutralizing activity. Suitable examples of fragments that may be used include, but are not limited to, Fab or F(ab′)hd 2, and Fv fragments.

Methods suitable for the generation and/or identification of antibodies capable of binding specifically to a target such as E-cadherin are well known to those skilled in the art. In general suitable antibodies may be generated by the use of isolated E-cadherin as an immunogen. E-cadherin may be administered to a mammalian organism, such as a rat, rabbit or mouse and antibodies elicited as part of the immune response. Suitable immunogens may include the full-length E-cadherin or an antigenic peptide fragment thereof (such as a preferred epitope associated with E-cadherin's biological function). Monoclonal antibodies capable of neutralizing E-cadherin activity can be produced by hybridomas, immortalized cell lines capable of secreting a specific monoclonal antibody. Suitable immortalized cell lines can be created in vitro by fusing two different cell types, usually lymphocytes, one of which is a tumour cell.

Further examples of suitable inhibitors of E-cadherin activity that may be used in accordance with the present invention may comprise proteins (or protein derivatives) able to bind to E-cadherin and thereby prevent its biological activity. Such proteins or derivatives include naturally occurring proteins able to inhibit E-cadherin activity, as well as derivatives based on such naturally occurring proteins, and novel proteins or derivatives possessing suitable activity.

For example, it is well known that E-cadherin binds to other E-cadherin molecules via the most terminal CAD extracellular domain (CAD-HAV). Similarly, it has been shown that tryptophan residue Trp156 is linked to dimerisation of E-cadherin. Accordingly, suitable inhibitors of E-cadherin activity for use in accordance with the present invention may include protein or other binding molecules capable of binding the CAD-HAV sequence or a sequence incorporating residue Trp156. Preferred inhibitors of E-cadherin activity may comprise the CAD-HAV sequence, and a suitable example of such an inhibitor of E-cadherin activity consists of the CAD-HAV sequence. Suitable inhibitors may comprise soluble E-cadherin fragments incorporating CAD-HAV and/or Trp156. Alternatively suitable protein or other binding molecules for use as inhibitors of E-cadherin activity in accordance with the present invention may be based on modified forms of the CAD-HAV sequence, or a sequence incorporating Trp156. Such modified forms may include derivatives that are modified in order to increase their biological activity, increase their resistance to protein degradation, increase their half-life, or otherwise increase their availability.

Suitable peptide inhibitors comprising the CAD-HAV sequence or Trp156 may comprise three or more contiguous amino acids from the sequence of E-cadherin shown in SEQ ID NO. 4, or may comprise five, ten, twenty or more contiguous amino acid residues from SEQ ID NO. 4 including the CAD-HAV sequence or Trp156.

Peptide inhibitors (such as those comprising the CAD-HAV sequence and/or sequences incorporating Trp156) may constitute suitable inhibitors of E-cadherin activity for use in accordance with the invention. Other suitable inhibitors of E-cadherin activity may be derived from such peptide inhibitors. Derivatives of this sort, such as peptoid derivatives, may have greater resistance to degradation, and may thus have improved shelf-lives compared to the peptides from which they are derived.

Suitable inhibitors of E-cadherin activity may also be conjugated with polyvalent/monovalent synthetic polymers, thereby increasing avidity of the inhibitors to their target protein. For example, in a suitable embodiment, multiple forms of inhibitors suitable for use in accordance with the invention may be conjugated to a single polymer. Alternatively or additionally a suitable inhibitor may be conjugated to a suitable polymer in combination with one or more other factors required to maintaining pluripotency (e.g. suitable oligosaccharides).

Inhibitors of E-cadherin activity suitable for use in accordance with the invention may alternatively, or additionally, be capable of binding to the membrane proximal region of E-cadherin.

Further inhibitors of E-cadherin activity suitable for use in accordance with the present invention include the α_{E}β₇ integrin, which is a naturally occurring binding partner of E-cadherin. Other suitable inhibitors may include E-cadherin-binding fragments of α_{E}β₇ integrin, or derivatives of this integrin or its fragments. Suitable fragments may be selected in the light of the disclosure of Shiraishi et al, (J Immunol. 2005 Jul. 15; 175(2):1014-21).

Small molecule inhibitors of E-cadherin may represent suitable inhibitors for use in accordance with the present invention.

In a suitable embodiment of the invention cells may be induced to over-express naturally occurring inhibitors of E-cadherin activity. It may be preferred that such over expression of naturally occurring inhibitors by a cultured cell is achieved transiently, and ceases once neural precursor cells, or neural cells, have been produced.

One example of such a naturally occurring inhibitor of E-cadherin activity is “Slug” (which is also known as “Snai2” and “snail homolog 2”). The amino acid sequence of the human form of Slug (NCBI reference number NPJ303059) is shown in SEQ ID NO. 5, and the amino acid sequence of the mouse form of Slug (NCBI reference number NP_{—}035545) is shown in SEQ ID NO. 22.

Another example of a suitable naturally occurring inhibitor of E-cadherin activity is “Snail”. The amino acid sequence of the human form of Snail (NCBI reference number NP_{—}005976) is shown in SEQ ID NO. 6, and the amino acid sequence of the murine form of snail (NCBI reference number NP_{—}035557) is shown in SEQ ID NO. 7.

A further naturally occurring inhibitor of E-cadherin activity suitable for use in accordance with the present invention comprises SMAD interacting protein 1 “SIP1”. The amino acid sequence of the human form of SIP1 (NCBI reference number BAB40819) is shown in SEQ ID NO. 8, and the amino acid sequence of the mouse form of SIP1 (NCBI reference number AAD56590) is shown in SEQ ID NO. 9.

E2A comprises a further naturally occurring inhibitor of E-cadherin activity suitable for use in accordance with the present invention. The human form of E2A is also known as “Homo sapiens transcription factor 3”, “E2A immunoglobulin enhancer binding factors E12/E47” and “TCF3”. The human form of E2A has been given NCBI reference number NM_{—}003200. The amino acid sequence of human E2A is shown in SEQ ID NO. 10, and DNA encoding the human form of E2A is shown in SEQ ID NO. 11. The murine form of E2A is also known as “Mus musculus transcription factor E2a” and has NCBI reference number BC006860. The amino acid sequence of murine E2A is shown in SEQ ID NO. 12, and the sequence of DNA encoding the murine form of E2A is shown in SEQ ID NO. 13.

It will be appreciated that the naturally occurring inhibitors of E-cadherin described above merely represent examples of the range of naturally occurring inhibitors that may be used in accordance with the invention. These (and other) inhibitors may be used singly or in combination with other inhibitors (including combinations of naturally occurring and artificial inhibitors).

The inventors believe that Snail, Slug, SIPI and E2A inhibiting E-cadherin expression by methylation/hypermethylation of the E-cadherin promoter, thus preventing or reducing gene transcription. Accordingly, agents capable of causing methylation or hypermethylation of the E-cadherin promoter represent suitable inhibitors of E-cadherin suitable for use in accordance with all aspects of the present invention. It will be appreciated that once such agents have caused methylation or hypermethylation of the E-cadherin promoter they need no longer be provided to cells.

Aptamers comprise a further example of preferred inhibitors of E-cadherin activity suitable for use in accordance with the present invention. Aptamers are nucleic acid molecules that that assume a specific, sequence-dependent shape and bind to specific target ligands based on a lock-and-key fit between the aptamer and ligand. Accordingly suitable aptamers may be designed to interact with E-cadherin protein or with nucleic acids encoding E-cadherin. Typically, aptamers may comprise either single- or double-stranded DNA molecules (ssDNA or dsDNA) or single-stranded RNA molecules (ssRNA).

As indicated above, aptamers may be used to bind (and thereby inhibit) E-cadherin protein and/or nucleic acids encoding E-cadherin protein. ssDNA aptamers may be preferred for use in the investigation of nucleic acids encoding E-cadherin.

Suitable aptamers may be selected from random sequence pools, from which specific aptamers may be identified which have suitably high affinity for E-cadherin protein or nucleic acid targets. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction.

The use of aptamers as inhibitors of E-cadherin activity in accordance with the present invention may be advantageous, since aptamers have relatively stable shelf lives. This may be particularly preferred in association with cell culture media of the invention. Aptamers suitable for use in accordance with the invention may be stabilized by chemical modifications (for example 2′-NH2 and 2′-F modifications).

Although the inventors do not wish to be bound by any hypothesis, it is believed that certain inhibitors, such as the antibody DECMA-I mentioned above, achieve their effect through the internalisation of E-cadherin. Such internalised protein cannot achieve its normal biological function, and so biological activity is thereby inhibited. Accordingly agents capable of causing the internalisation of E-cadherin represent suitable inhibitors for use in accordance with the invention.

The preceding examples have concentrated primarily on inhibitors able to prevent biological activity that may otherwise be associated with E-cadherin that has already been expressed. It will be appreciated that other suitable inhibitors may include agents capable of preventing the expression of E-cadherin. Such inhibitors may prevent or reduce transcription of the E-cadherin gene, or may prevent or reduce translation of E-cadherin gene transcripts.

Examples of such inhibitors capable of preventing the expression of E-cadherin include aptamers (as considered above), antisense oligonucleotides and ribozymes. Suitable inhibitors will also encompass agents that can disrupt the E-cadherin gene.

The skilled person will realise that many of the inhibitors of E-cadherin activity described in the present specification, and particularly protein or nucleic acid agents as described herein, are suitable for cellular production (using the mechanism of gene transcription and expression). The skilled person will recognise that preferably such agents may be produced by the cells from which neural progenitor cells are to be produced. Suitably such agents may be provided in a genetic construct that is transiently incorporated, or transiently expressed, in or by the cells. The inhibitor of E-cadherin activity encoded by the construct may preferably comprise an siRNA molecule, such as those set out in SEQ ID NOS. 14-21.

It will be appreciated from the above that the inhibitors of E-cadherin activity that may be used in the methods of the invention include exogenous inhibitors of E-cadherin activity (such as peptides, antibodies, or the like) and endogenous inhibitors of E-cadherin activity (such as siRNA molecules). In the case that it is desired to use endogenous inhibitors in the various aspects of the present invention, it may preferred that these are “direct” inhibitors, as opposed to “indirect” inhibitors that compete for factors involved with E-cadherin function.

The use of exogenous inhibitors of E-cadherin activity in the methods of the invention may provide advantages in that they reduce the extent to which it is necessary to genetically manipulate cells from which neural precursor will be produced. Modifications of such cells associated with the expression of endogenous inhibitors may be expected to remain in both the cells having neural potential and in the neural precursor cells. It may be preferred to avoid such modifications in circumstances in which the neural precursors (or their neural cell progeny) will be provided to a host, for example in therapeutic applications. Use of exogenous inhibitors of E-cadherin activity may also facilitate better control of the amount of the inhibitor provided, since one practicing the methods of the invention will be able to accurately determine the amount of the inhibitor provided.

Particularly suitable examples of inhibitors of E-cadherin activity that the inventors have found to be particularly effective in practicing the methods of the invention are the inhibitory peptide SWELYYPLRANL (SEQ ID NO. 1), and its derivatives H-SWELYYP-NH₂ (SEQ ID NO. 2) or SWELYYPL (SEQ ID NO. 26). This inhibitor of E-cadherin activity is suitable for use as an exogenous inhibitor provided in the cell culture medium. Fragments or derivatives of this peptide that retain the ability to inhibit E-cadherin activity may also be used in the methods of the invention. It may generally be preferred to employ the peptide of SEQ ID NO. 1, as opposed to its derivatives.

It will be appreciated that expression of E-cadherin need not be inhibited (either totally or partially) in order to practice the methods of the invention. The inventors believe that the methods of the invention may be effectively practiced using inhibitors that reduce transhomodimerisation of E-cadherin, which is associated with E-cadherin activity. Agents capable of reducing transhomodimerisation of E-cadherin may thus represent preferred inhibitors of E-cadherin for use in the various aspects of the invention.

In suitable embodiments utilising human cells and the peptide inhibitor SWELYYPLRANL (SEQ ID NO. 1) described above, the inhibitor may be added to cell culture medium such that a 500 μM solution of the inhibitor is produced. In suitable embodiments utilising murine cells and the peptide inhibitor SWELYYPLRANL the inhibitor may be added to cell culture medium such that a 1 mM solution of the inhibitor is produced.

The inventors have found that when exogenous inhibitors of E-cadherin activity are used, these inhibitors may be provided to the cells transiently. In suitable embodiments, an inhibitor of E-cadherin activity may be provided to cells for a period of up to 14 days, up to 12 days, up to ten days, up to eight days, up to six days, or up to four days. Merely by way of example, in the Experimental Results that follow, neural precursor cells are efficiently produced in methods in which the peptide inhibitor SWELYYPLRANL (SEQ ID NO. 1) is provided to cells every two days for six to seven days after induction of stress in cells, but that no further inhibitor need be added for the remaining period during which neural precursors cells are generated and cultured.

This provides important advantages in that it reduces the total amount of such inhibitors that need to be provided over the course of methods of the invention, which may be beneficial since such inhibitors may be expensive. Furthermore, the finding that only a relatively short period of inhibition is needed is consistent with the desirable aim of reducing factors provided to cells that may be re-introduced to a patient (for example as part of a therapy).

In a fifth aspect of the invention there is provided a neural precursor cell produced by a method in accordance with the invention.

In a sixth aspect of the invention there is provided a neural cell produced by a method in accordance with the invention.

In a seventh aspect of the invention there is provided a glial cell produced by a method in accordance with the invention.

In an eighth aspect of the invention there is provided a neuronal cell produced by a method in accordance with the invention.

It will be appreciated that any of the cells considered in the various aspects of the invention may incorporate modifications, such as modifications associated with adaptation for experimental or therapeutic use, that allow them to be distinguished from naturally occurring cells of an otherwise corresponding type.

Merely by way of example, cells in accordance with the aspects of the present invention may incorporate a modification in which one or more therapeutically relevant genes have been modified, such that expression of the gene(s) in question is/are altered.

Cells in accordance with the aspects of the invention may, additionally or alternatively, incorporate a modification in which one or more genes associated with an activity or phenotype characteristic of a disease state have been modified, such that expression of the gene(s) in question is/are altered. This alteration may allow the cells to replicate certain activities or phenotypes of cells associated with the disease state in question. As a consequence, cells of the invention modified in this manner may be used in the screening or identification of agents that influence (either ameliorating or exacerbating) the disease state. Thus cells in accordance with the invention may be used in the development or identification of novel therapeutic agents.

The invention will now be further described with reference to following Experimental Results, and the accompanying Figures, in which:

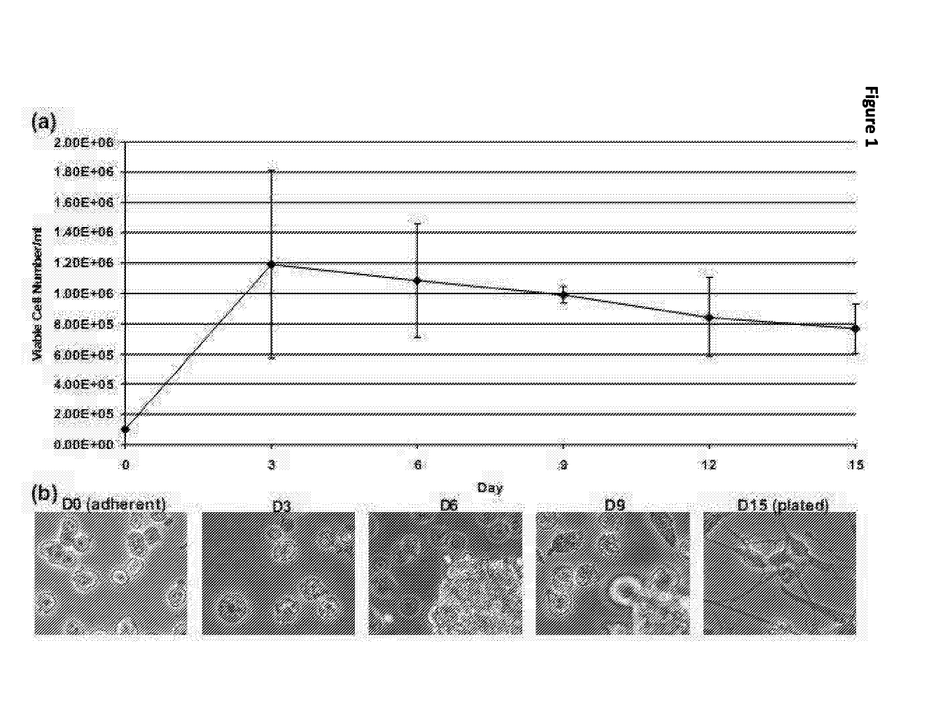

FIG. 1 illustrates differentiation of ENPS cells towards neural lineages in shake flask suspension culture. Briefly, undifferentiated ENPS cells were maintained under standard adherent culture conditions prior to shake flask culture. ENPS cells were seeded into shake flasks at 1.0E5 vc/ml in 25 ml of differentiation media in 125 ml shake flasks and agitated at 140 rpm for 15 days. Cell counts and media replenishment were performed every 72 h. (a) Total viable cell numbers peaked following 3 days in culture (mean viability 77%). However, from day 3 onwards a significant decrease in cell viability was observed (mean viability was 21±7% for the duration of the experiment). Values represent mean±SEM, n=3. (b) Phase contrast microscopy shows that cells maintained in shake flask cultures have dispersed growth, and at day 6, formation of cell spheres are observed. At day 15 cells were transferred to gelatin-treated plates and allowed to adhere overnight prior to analysis. These cells exhibit typical culture morphology associated with neural cell lineages.

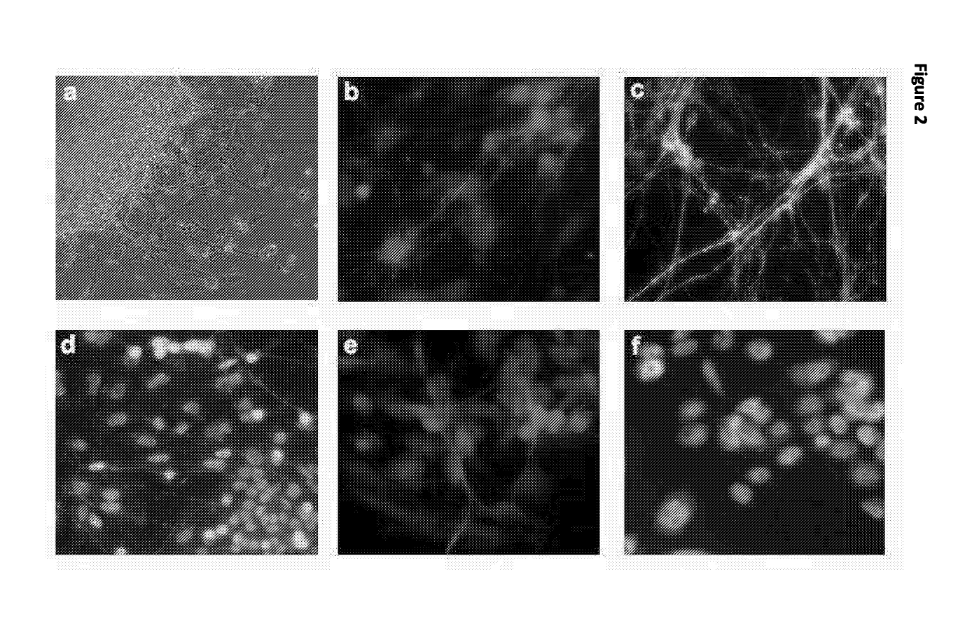

FIG. 2 shows characterisation of ENPS cells differentiated towards neural lineages in shake flask suspension culture. In this study, ENPS cells were cultured in differentiation media (knockout DMEM supplemented with 10% serum (3:7 parts FBS:KSR), 2 mM L-glutamine, non-essential amino acids (100×, 1:100 dilution), 50 μM 2-mercaptoethanol at 37° C./5% CO₂) in shake flask suspension culture at 140 rpm over 15 days. Cells were harvested on day 15 and plated onto gelatin coated dishes and allowed to adhere overnight. Phase contrast (a) and immunofluorescent analysis of markersrepresentative of neural lineages (b) Nestin (red), (c) _III-Tubulin (green), (d) NeuroD-1 (green), (e) Neurofilament (red) and (f) Pax6 (green). Total cells were visualised using DAPI (blue). All images captured at ×20 magnification.

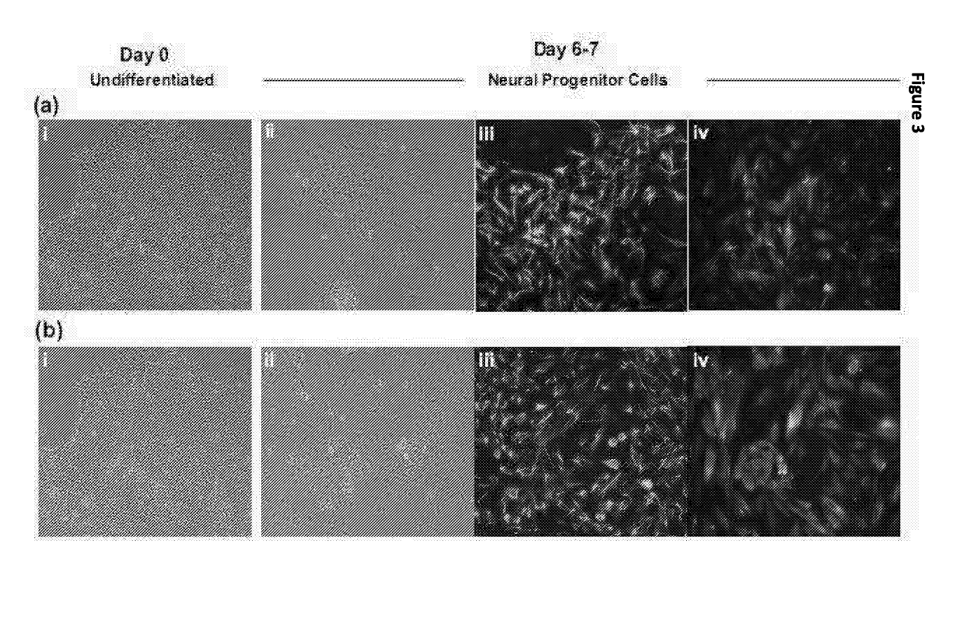

FIG. 3 illustrates differentiation of human ES cells towards neural lineages in adherent culture. Human ES cells (HUES7) were grown under standard adherent feeder-free culture conditions prior to induction of differentiation (ai) and (bi). Confluent undifferentiated cells were dissociated and seeded at a low density (2.0E5 cells/962 mm2) onto gelatin coated wells in (a) differentiation medium alone or (b) media supplemented with peptide (500 μM). Media (and peptide) were replenished every 2 days and cells split accordingly to maintain <70% confluence. Phase contrast images show the majority of cells cultured for 6-7 days in (aii) media alone, exhibit a flattened and ‘jagged’ morphology (concomitant with differentiating cells), however few colonies of undifferentiated cells remain. Cells cultured for 6-7 days in (bii) media supplemented with peptide, exhibit a similar morphology, however no undifferentiated colonies were observed. To identify the phenotype of these cells, cultures were harvested at day 7 and immunofluorescent analysis of neural progenitor cell markers was performed. (aiii) Cells differentiated in media alone show positive Nestin (green) and (aiv) Vimentin (green) expression in a large proportion of cells, however (biii) cells cultured in the presence of peptide show homogenous expression of Nestin and (biv) Vimentin. Total cells were visualised using DAPI (blue). All images were captured at ×10 magnification.

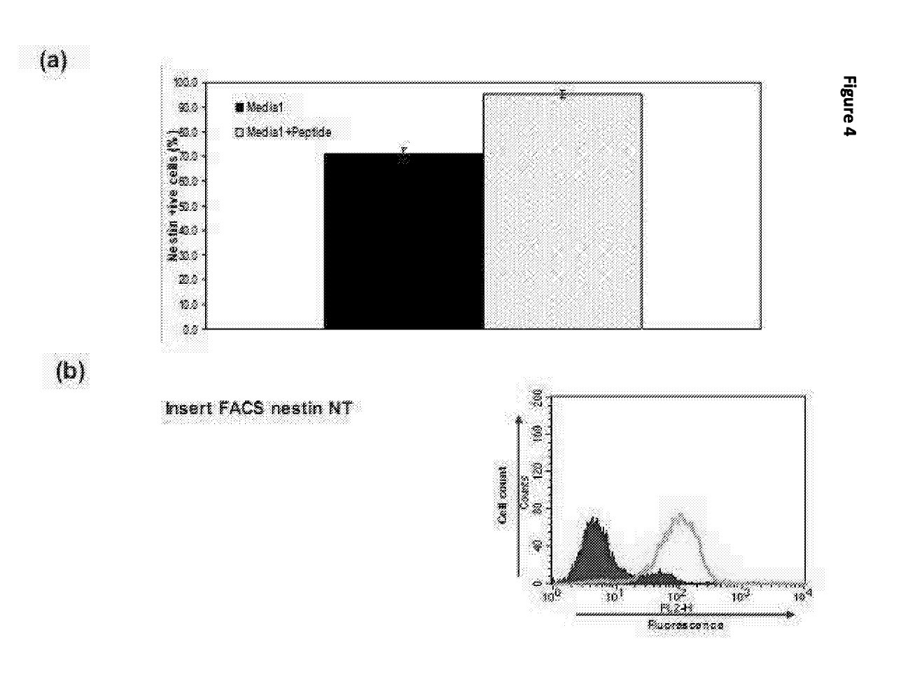

FIG. 4 shows details of characterisation of human ES cells differentiated towards neural lineages in adherent culture. To identify the phenotype of these cells, cultures were harvested at day 7 and immunofluorescent analysis of neural progenitor cell markers was performed. (a) Quantification of the number of Nestin positive cells cultured in media alone was 71.1%, compared to 95.3% in media supplemented with peptide following 7 days of differentiation (n=3). (b) Human ES-derived neural progenitor cells are able to self renewal for extended periods of time (90 days) when cultured in the presence of 8 ng/ml fgf2 as shown by (d) fluorescent flow cytometry analysis of nestin expression. Nestin (green line profile) and isotype control antibody (filled purple profile).

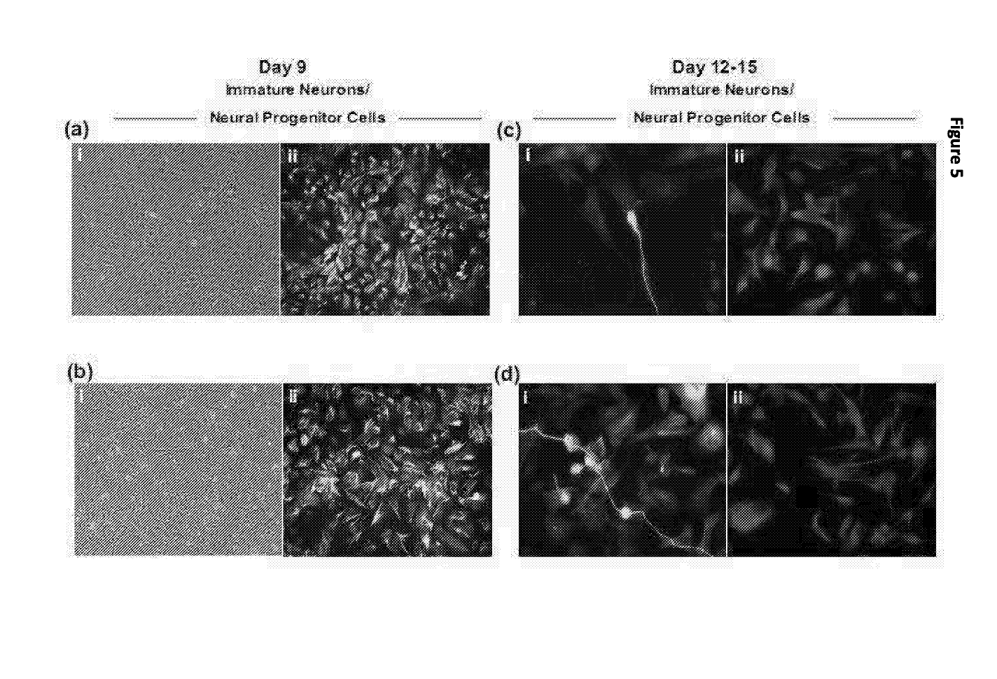

FIG. 5 sets out further details of characterisation of human ES cells differentiated towards neural lineages in adherent culture. Human ES cells differentiated under adherent culture conditions in (a) media alone or (b) media supplemented with peptide. Phase contrast images show typical morphology associated with neural cell lineages in cells grown in (ai) media alone and (bi) peptide-supplemented media on day 9. Cultures were harvested at day 9 for dual immunofluorescent analysis, both cells grown in (aii) in media alone and (bii) in peptide-supplemented media express Nestin (red) and neuron-specific β-III Tubulin (green), whereby a small proportion of negative cells (blue) are identified in (aii). Immunofluorescent image analysis of cells differentiated for 12-15 days in both (ci & cii) media alone and (di & dii) in peptide-supplemented media express markers of neural commitment; (ci & di) β-III Tubulin (green) and (cii & dii) Neurofilament (red). Total cell were visualised using DAPI (blue). Images captured at ×10 or ×20 magnifications.

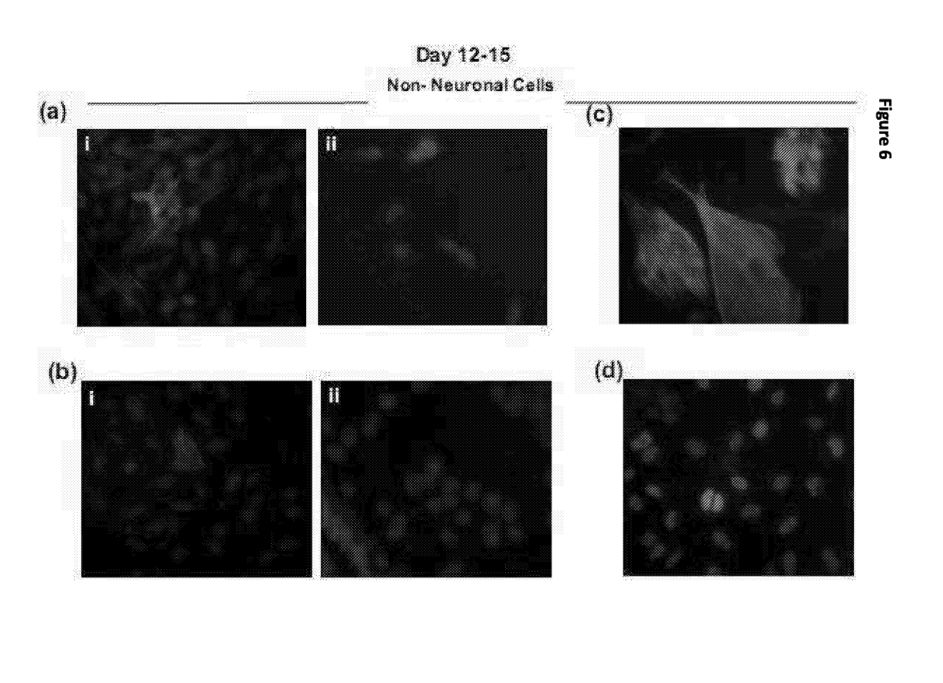

FIG. 6 shows assessment of non-neuronal lineages in directed neural differentiation of human ES cells. Differentiated cells harvested on day 15 were stained in parallel with markers associated with non-neuronal lineages to assess. Small populations of cells grown in (ai) media alone exhibited α smooth muscle actin (mesoderm) expression (red), however, cells cultured in (bi) media supplemented with peptide, α smooth muscle actin expression was only detected in 1 cell out of all cultures assessed (n=3). Cells differentiated for 15 days in (aii) media alone and (bii) in peptide-supplemented media showed no positive staining of the endoderm marker Forkhead box protein A2 (foxA2—green). (c&d) Adherent undifferentiated cells were induced to differentiate by overgrowing for 15 days to serve as a positive control for three lineages. Immunofluorescent analysis of these cells in parallel show (c) extensive a smooth muscle actin expression (red) and (d) positive nuclear immunoreactivity of foxA2 (green) in positive control samples. Total cells were visualised using DAPI (blue).

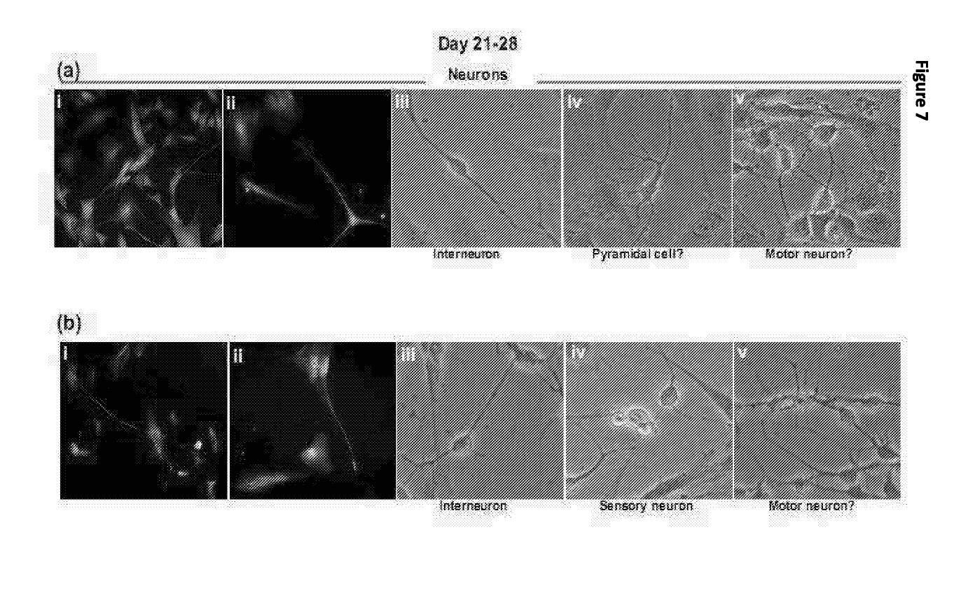

FIG. 7. Human ES cells cultured in (a) media alone or (b) peptide-supplemented media (for 7 days) were differentiated (using a specialty media*) towards neuronal lineages. Cells were harvested on days 21 and 28 for and assessed for markers of neurons. (ai&bi) Positive dual immunoreactivity of Neurofilament (red) and βIII-Tubulin (green) at day 21 and (aii&bii) MAP2 (green) at day 28. Total cells were visualised using DAPI (blue). (iii-v) Phase contrast images of neuron types (day 21-31).

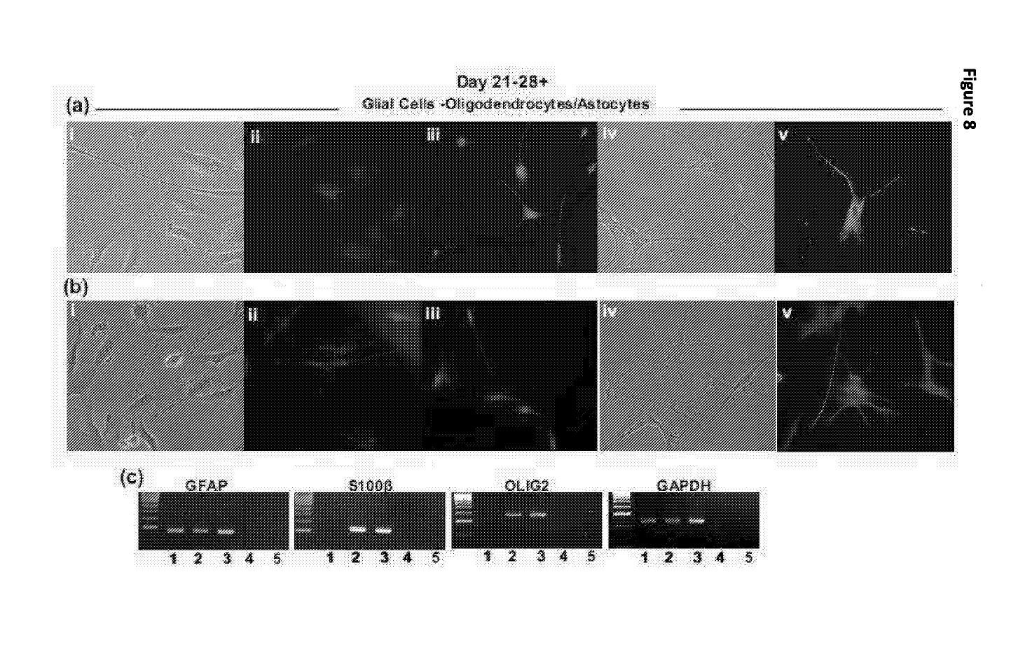

FIG. 8. Human ES cells cultured in (a) media alone or (b) peptide-supplemented media (for 7 days) were differentiated (using a specialty media*) towards glial lineages. Cells were harvested on days 21 and 28 for and assessed for markers of glial cell subsets (i) Phase contrast images of astrocytes (day 21). (ii) Positive immunoreactivity of A2B5 (red) (early astrocyte marker) at day 21 and (iii) GFAP (red) (pan astocyte marker) at day 28. (iv) Phase contrast images of oligodendrocyte-like cells. (v) Positive immunoreactivity of O4 (green) (oligodendrocyte progenitor marker) at day 21. Total cells were visualised using DAPI (blue). (c) RT-PCR analysis was performed on cells cultured for 21 days in glial-differentiation media*. (1) Media1 alone (2) Media1+peptide (3) Positive (serum) control (4) Negative control (−RT) (5) Negative (no template control).

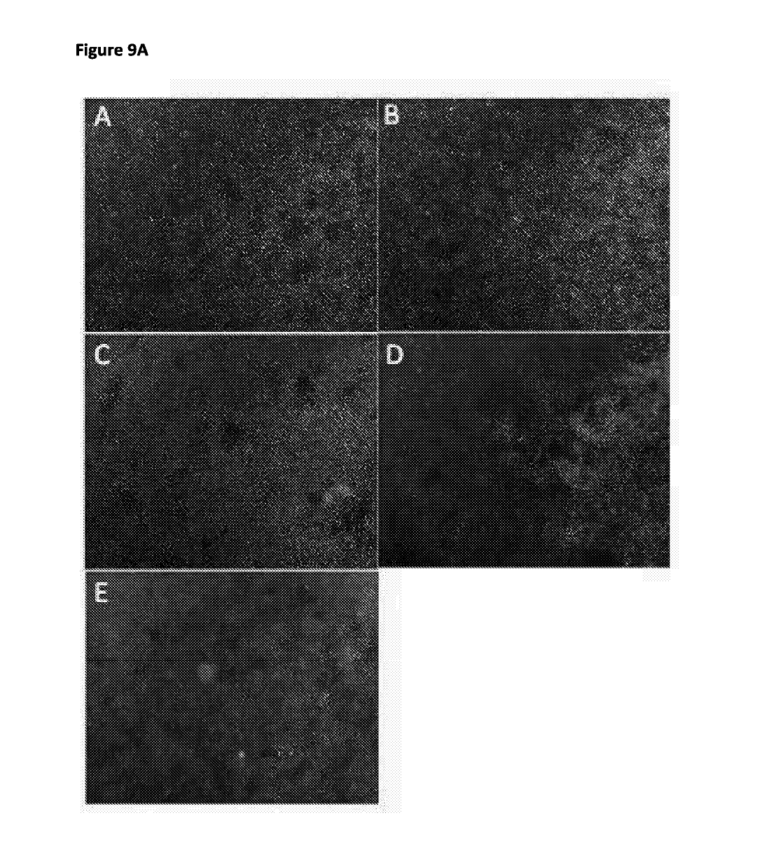

FIG. 9. This Figure illustrates the effect of E-cadherin on cell-cell contact in pluripotent hiPSCs. Human iPS cells were cultured in MTesR complete media under standard adherent feeder free conditions supplemented with either; (A) peptide A, (B) E-cadherin neutralising antibody, (C) peptide C, (D) peptide B and (E) control (water only) for 48 h. Phase contrast images show that loss of cell-cell contact is achieved in the majority cells (>85%) when cultured with peptide A and E-cadherin neutralising antibody (A&B respectively), compared to the typical compacted ‘colony’ morphology of hiPSCs (shown in E). In contrast, cells cultured in the presence of peptide B retain the compacted morphology typical of hiPSCs (1D). The culture of hiPSCs in media supplemented with peptide C shows loss of cell-cell contacts in approx. 50-60% of the cell population, however this is markedly lower when compared to peptide A or neutralising antibody.

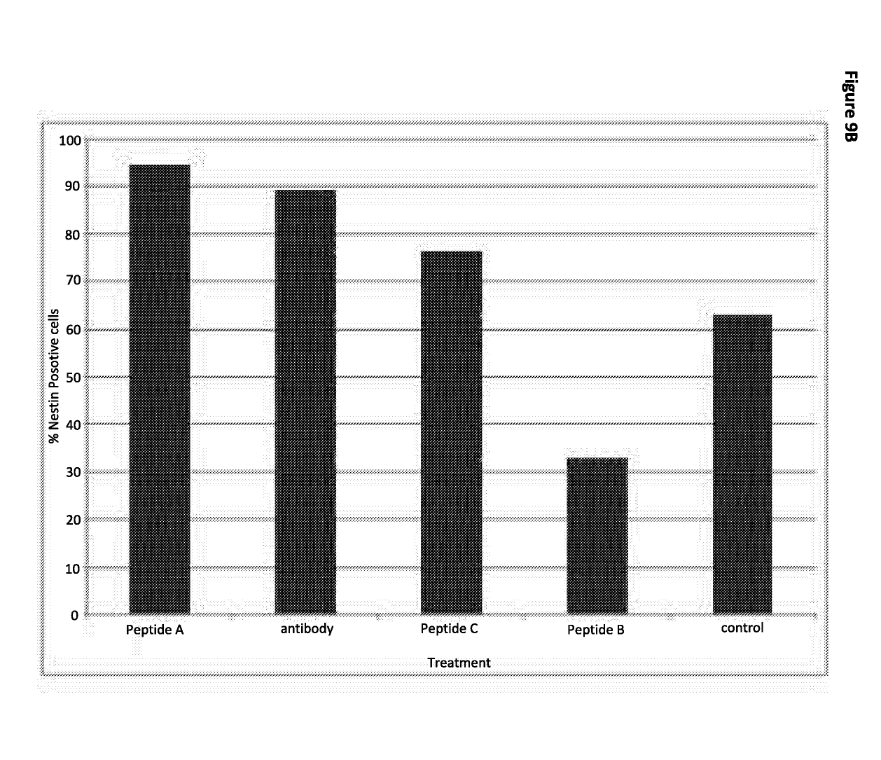

FIG. 10. This Figure illustrates neural differentiation of hiPSCs using E-cadherin inhibitors. To initiate differentiation confluent undifferentiated hiPS cells were dissociated and seeded at a low density onto gelatin coated wells in differentiation medium supplemented with/without E-cadherin-inhibitors for 7 days. To identify the phenotype of these cells, cultures were harvested at day 7 and immunofluorescent analysis of the neural progenitor cell marker Nestin was performed. Cells were treated daily for 7 days with (A) peptide A, (B) neutralising antibody, (C) peptide C, (D) peptide B and (E) control. Low power magnification (×10) shows distribution of Nestin positive cells (green) with total nuclei stained using DAPI (blue). (II) Quantification of the number of Nestin positive cells cultured in media was maximal when cells were cultured in peptide A (94%), compared to media supplemented with neutralising antibody (89%), peptide C (76%), peptide B (33%) and control (63%).

### EXPERIMENTAL RESULTS STUDY 1

### 1 Materials and Methods

### 1.1 Adherent Culture of Mouse ENPS Cells

Mouse E-cadherin negative pluripotent stem cells (ENPS) cells were derived by Dr Ward (unpublished data) and cultured on gelatin-treated plates in knockout Dulbecco's modified Eagle's medium (DMEM) supplemented with 10% fetal bovine serum (FBS), 2 mM L-glutamine, nonessential amino acids (NEAA) (1×), and 50 μM 2-mercaptoethanol (all from Invitrogen) and 1,000 units/ml LIF (ESGRO; Millipore) at 37° C. and 5% CO₂ unless otherwise stated. The medium was replenished every 48 hours and cells passaged prior to confluence (2 days). Gelatin treated plates were made by the addition of 0.1% w/v gelatin (Sigma) in sterile ddH₂0 to tissue culture treated plates (Griener-Bio) and incubated overnight at 4° C.

### 1.2 Suspension Culture of Mouse ENPS Cells

Mouse ENPS cells were dissociated from adherent culture using Trypsin-EDTA (Sigma) and seeded into shake flasks at 1.0E5 viable cells/ml (vc/ml) in 25 ml of differentiation media (Knockout DMEM supplemented with 10% serum (3:7 parts FBS:KSR), 2 mM L-glutamine, non-essential amino acids (100×, 1:100 dilution), 50 μM 2-mercaptoethanol) in 125 ml Erlenmeyer shake flasks (Corning) and agitated at 140 rpm on a shaking platform at 37° C./5% CO2 (1″ orbit—140 rpm; Satorius, Surrey, UK) for 15 days. Cell counts and media replenishment were performed every 72 h.

### 1.3 Maintenance of Undifferentiated Human ES Cells in Feeder-Free Adherent Culture

Human ES cell lines, HUES7 (passage 39-44), H9 (passage 50-55) and MAN7 (passage 18-21) were grown under adherent feeder-free culture conditions prior to induction of differentiation. Cells were cultured in STEMPRO® (Invitrogen—complete medium) which comprises; DMEM/F-12+GlutaMAX, 8 ng/ml FGF-basic factor (Peprotec), STEMPRO® hESC SFM Growth Supplement (1×), 1.8% BSA, and 0.1 Mm 2-mercaptoethanol. Cells were cultured on either Matrigel™- (BD Biosciences 356234) or Geltrex™- (Invitrogen 12760-021) coated tissue culture grade plates. Matrigel-treated plates were coated with pre-diluted Matrigel™ (1:100 in DMEM/F12 media) and incubated at room temperature prior to use. Geltrex™-coated plates were coated with pre-diluted Geltrex™ (1:29 in DMEM/F12 media) and incubated for 1 h at 37° C. prior to use. Media was replenished every 24 h and cells were passaged upon confluency. All cells were propagated for a minimum of two passages as feeder-free cultures to exclude unwanted residual mouse fibroblast feeder cells. Cells were dissociated either using trypsin-EDTA (Sigma) or Collagenase IV (Sigma-1 mg/ml final concentration) dependent on the ES cell line used.

### 1.4 Differentiation of Human ES Cells in Adherent Culture

Confluent undifferentiated cells were dissociated and seeded at a low density (2.0E5 cells/962 mm2) onto 0.1% w/v gelatin coated wells in differentiation medium alone (DMEM/F-12+GlutaMAX (Invitrogen), 10% Knockout Serum Replacement (KSR) (Invitrogen), Penicillin/Streptomycin (1×) (PAA) for 24 hours prior to media supplementation with an E-cadherin inhibiting peptide (H-Ser-Trp-Glu-Leu-Tyr-Tyr-Pro-Leu-Arg-Ala-Asn-Leu-NH₂, >95% purity, acetate salt background) (Bachem) as published in Devemy & Blashuk (2009). Peptide was reconstituted at 30 mg/ml in sterile ddH₂O (20 mM stock concentration), with a working concentration 500 μM for inhibition of human E-cadherin. Media (and peptide) were replenished every 2 days for 6-7 days. After this time peptide is no longer necessary. Morphological analysis and immunostaining with markers for neural precursor cells and more mature neural cells were performed during the course of the differentiation protocol.

### 1.5 Propagation of Human Neural Precursor Cells

To maintain self-renewal of neural precursor cells (NPCs), cultures from day 7 onwards were transferred to fresh gelatin coated plates and cultured in expansion media (DMEM/F12 Glutamax, 10% FBS (both Invitrogen), 8 ng/ml FGF basic factor (Peprotec), Penicillin/Streptomycin (1×) (PAA). Media were replenished every 2-3 days and cells were split accordingly to maintain <70% confluence.

1.6 Differentiation into Mature Neural and Glial Restricted Lineages

Immature neurons/NPCs were differentiated using established protocols cell culture media commercially available from Invitrogen. Briefly, confluent NPCs (4.5-5.5×10⁵/962 mm2) were dissociated using trypsin EDTA and re-plated in 0.1% w/v gelatin treated 6-well plates (unless otherwise stated) in the relevant differentiation media. Media were replenished every 2/3 days. Cultures were propagated for >21 days. In addition, to serve as a positive control for all three somatic lineages, undifferentiated human ES cell cultures were induced to spontaneously differentiate by high-confluent culture in the presence of 10% FBS.

### 1.6.1 Glial Differentiation

### 1.6.1.1 Astrocyte Differentiation

Confluent NPCs (4.5-5.5×10⁵/962 mm2) were dissociated using trypsin EDTA and re-plated in Geltrex™-coated 6-well plates and cultured in DMEM+GlutaMAX, N2, 1% FBS, Penicillin/Streptomycin (1×) (PAA). Media were replenished every 2/3 days. Cultures were propagated for >21 days.

### 1.6.1.2 Oligodendrocyte Differentiation

Confluent NPCs (4.5-5.5×10⁵/962 mm2) were dissociated using trypsin EDTA and re-plated in Geltrex™-coated 6-well plates and cultured in Neurobasal media, B27 (1×), stable glutamine (1×) (all Invitrogen), T3 (30 ng/ml—Sigma), Penicillin/Streptomycin (1×) (PAA). Media were replenished every 2/3 days. Cultures were propagated for >21 days.

### 1.6.2 Neural Differentiation

Tissue culture grade plates were pre-coated using poly-L-ornithine (Sigma—20 μg/mL) overnight at room temperature. Excess poly-L-ornithine was removed and plates were coated with laminin for 4 h at 37° C. (Invitrogen—10 μg/mL) prior to cell culture. Confluent NPCs (4.5-5.5×10⁵/962 mm2) were dissociated using trypsin EDTA and re-plated in 10 μg/ml laminin treated 6-well plates and cultured in Neurobasal® media, B27 (1×), stable glutamine (1×), Non-essential amino acids (1×) (all Invitrogen), Penicillin/Streptomycin (1×) (PAA). Media were replenished every 2/3 days. Cultures were propagated for >21 days.

### 1.7 Immunofluorescent Imaging

Cells were fixed in 4% w/v paraformaldehyde and stained in situ (Mohamet et al, 2010). Primary antibodies were as follows; mouse anti-NESTIN (1:250), mouse anti-neuron specific β-III TUBULIN (β-III TUB) (1:1000) mouse anti-NEUROD1 (1:00), rabbit anti-PAX6 (1:100), mouse anti-α SMOOTH MUSCLE ACTIN (ASMA) (1:50), goat anti-FOXA2 (1:50), mouse anti-VIMENTIN (1:20), rabbit anti-MAP2 (1:200), mouse anti-A2B5 (1:500), chicken anti-GFAP (1:500) (All Abcam, Cambridge, UK), rabbit anti-NEUROFILAMENT (1:500) (Enzo Life Sciences) and mouse anti-O4 (1:500) (R&D Systems). The appropriate secondary antibodies conjugated with Alexa Fluors 488 or 546 were used (1:500, Invitrogen, Paisley, UK) and all samples were mounted using DAPI Vector shield (Vector Laboratories, Peterborough, UK). The cells were viewed on a Leica DM500 fluorescence microscope.

### 1.8 Fluorescent Flow Cytometry Analysis

Cells were dissociated from adherent culture using dissociation buffer (Invitrogen, Paisley, UK). Briefly, the cells were washed in PBS and fixed in 1% w/v paraformaldehyde for 10 mins at room temperature, followed by cell permeation using 70% v/v ice cold methanol at −20° C. for 30 mins. The cells were re-suspended in 0.2% w/v BSA in PBS containing the primary antibody, anti-mouse NESTIN (1:100 Abcam) or an IgG control isotype incubated for 30 min on ice. Cells were washed and resuspended in the appropriate phycoerythrin-conjugated secondary antibody (1:100 Santa Cruz Biotechnology) and incubated for 30 min on ice. The cells were washed and re-fixed in 1% w/v paraformaldehyde. Cell fluorescence was analysed using a Becton Dickinson FACScaliber. Viable cells were gated using forward and sidescatter and all data represents cells from this population.

### 1.9 RT-PCR

Total RNA was isolated from the cells using the RNeasy Kit, (Qiagen, West Sussex, UK) according to manufacturer's instructions. RNA preparations were quantified by absorbance at 260 nm (A₂₆₀) using a Nanodrop spectrophotometer (Labtech Intl., E. Sussex, UK). Synthesis of cDNA was performed using Applied Biosystems High capacity RNA to cDNA Kit as per manufacturer's instructions utilising 1 μg RNA (Invitrogen). PCR was performed using 1 μl of the cDNA and 35 cycles at the optimal annealing temperature. Samples were run on 2% w/v agarose gels containing 400 ng/ml ethidium bromide and visualized using an Epi Chemi II Darkroom and Sensicam imager with Labworks 4 software (UVP, CA, USA). Primer sequences and annealing temperatures were as set out in the table below.

| | | | Temperature |
|---|---|---|---|
| | Primer sequence | Primer sequence | (annealing) |
| Gene | 5′-3 (forward) | 5′-3 (reverse) | (° C.) |
| GFAP | TCATCGCTCAGGAGGTCCTT | CTGTTGCCAGAGATGGAGGTT | 60 |
| OLIG2 | GCTGTGGAAACAGTTTGGGT | AAGGGTGTTACACGGCAGAC | 60 |
| S100β | GCCATGGCCGTGTAGACCCT | ATCCCGGGAAGCAGGCCGAA | 60 |
| GAPDH | ACCCAGAAGACTGTGGATGG | TCTAGACGGCAGGTCAGGTC | 60 |

GFAP - Glial fibrillary acidic protein
OLIG2 - Oligodendrocyte transcription factor 2
S100β - S100 beta subfamily of EF-hand Calcium binding protein
GAPDH - Glyceraldehyde-3-phosphate dehydrogenase

### 2 Results

### 2.1 Differentiation of ENPS Cells in Manual Fed-Batch Shake Flasks

Undifferentiated ENPS cells were maintained under standard adherent culture conditions prior to suspension culture. Triplicate flasks were inoculated with 1×10⁵ vc/mL in 25 mL differentiation media and agitated at 140 rpm. The optimal cell seeding density was previously demonstrated in Mohamet et al (2010). Flasks were sampled every 72 h and viable cell number determined (FIG. 1a). Mean viable cell number peaked following 3 days in suspension culture (1.19×10⁶ vc/ml) decreasing to 7.65×10⁵ vc/ml over the 15 d culture period. This was also reflected in cell viability whereby, total cell viability peaked following 3 days in culture (mean viability 77±6.3%). However, from day 3 onwards a significant decrease in cell viability was observed (mean viability 21±7% for the duration of the experiment). Values represent mean±SEM, n=3. ENPS cells were cultured as described above, but without the addition of FBS, however, by day 3 the majority of cells were not viable. Phase contrast microscopy (FIG. 1b, ×20 magnification) shows that cells maintained in shake flask cultures have dispersed growth, and by day 6, formation of cell spheres are observed. At day 15 cells were transferred to gelatin-treated plates and allowed to adhere overnight prior to analysis. Culture morphology shows that these cells exhibit similar morphology to neural cell types, and as such are adherent and exhibit neuron-like processes.

### 2.2 Characterisation of Differentiated ENPS Cells

To determine if ENPS cells grown in manual fed-batch culture over 15 d were of a neural phenotype we examined expression of a number of markers of neuronal lineage. ENPS cells grown in shake flasks for 15 d were plated onto gelatin-coated plates and allowed to adhere for 24 h under routine culture conditions in differentiation media. Phase contrast images show neural-like processes projecting from the main cell body (sphere) forming fibre bundles (FIG. 2a). The differentiated phenotype of ENPS cells was validated at the protein level with positive immunoreactivity for NESTIN; β-III TUBULIN, NEURO-D1, NEUROFILAMENT, and PAX6 (FIGS. 2(b), (c), (d), (e) and (f) respectively). These results demonstrate that ENPS cells cultured in a manual fed-batch shake flask over 15 d express markers concomitant with neural cell types.

### 2.3 Differentiation of Human ES Cells in Adherent Culture Form Neural Precursor Cells

Human ES cells lines HUES7, H9 and MAN7 were grown under standard adherent feeder-free culture conditions prior to induction of differentiation and typical culture morphology is shown in FIGS. 3a and b. To initiate differentiation confluent undifferentiated cells were dissociated and seeded at a low density onto gelatin coated wells in differentiation medium alone (FIG. 3a) or media supplemented with E-cadherin-inhibiting peptide (FIG. 3b). Phase contrast images show the majority of cells cultured for 6-7 days in media alone, exhibit a flattened and ‘jagged’ morphology (concomitant with differentiating cells) however, few colonies of undifferentiated cells remain (FIG. 3aii). Cells cultured for 6-7 days in media supplemented with peptide, exhibit a similar morphology, however no undifferentiated colonies were observed (FIG. 3bii). To identify the phenotype of these cells, cultures were harvested at day 7 and immunofluorescent analysis of neural precursor cell markers was performed. Cells differentiated in media alone show positive NESTIN (green) (FIG. 3aiii) and VIMENTIN (green) (FIG. 3 aiv) expression in a large proportion of cells, however, cells cultured in the presence of peptide show homogenous expression of NESTIN and VIMENTIN (FIGS. 3biii and 3biv respectively). Total cells were visualised using DAPI (blue). Quantification of the number of NESTIN positive cells cultured in media alone was 71.1±2.2% compared to 95.3±% in media supplemented with peptide following 7 days of differentiation (FIG. 4a, n=3). Upon removal of peptide after 7 days in culture, the ES-derived neural precursor cells were propagated for a further 21 days in differentiation media alone. Phase contrast images show typical morphology associated with neural cell lineages in cells grown in media alone and peptide-supplemented media at day 9 (FIGS. 5ai and bi respectively). Cultures were harvested at day 9 for dual immunofluorescence of neuronal markers. Cells grown in media alone express NESTIN (red) and are beginning to express β-III TUBULIN (green), but a small proportion of negative cells (blue) can still be observed (FIG. 5aii). Cells derived in peptide-supplemented media express homogenous levels of NESTIN (red) and filamentous expression of β-III TUBULIN (green) (FIG. 5bii). Immunofluorescent image analysis of cells differentiated for 12-15 days in both media alone (FIGS. 5ci and cii) and in peptide-supplemented media (FIGS. 5di and dii) express markers of pan-neural commitment; neuron specific β-III TUBULIN (green) and Neurofilament (red) expression can be observed in neural precursor cells derived in media alone, however negative cells can be observed (FIGS. 5ci and cii respectively). Neural precursor cells derived in peptide-containing media express more mature filamentous expression of β-III TUBULIN (FIG. 5di) and NEUROFILAMENT (FIG. 5dii). Total cells were visualised using DAPI (blue).

2.4 Isolation of Neuronal Cells without Significant Contamination by Other Somatic Cell Types

Differentiated cells harvested on day 15 were stained in parallel with markers associated with non-neuronal lineages. Small populations of cells derived in media alone exhibited α smooth muscle actin (mesoderm) expression (red) (FIG. 6ai), however, in cells derived in media supplemented with peptide, a smooth muscle actin expression was only detected in 1 cell out of all cultures assessed (FIG. 6bi) (n=3). Furthermore, media or peptide-treated cells differentiated for 15 days showed no positive staining of the endoderm marker, Forkhead box protein A2 (foxA2—green) (FIGS. 6aii and bii respectively). Adherent undifferentiated cells were induced to differentiate by high-confluent culture in the presence of serum for 15 days to serve as a positive control for all three somatic cell lineages. Immunofluorescent analysis of these cells in parallel, demonstrates extensive a smooth muscle actin expression (red) (FIG. 6c) and positive nuclear immunoreactivity for foxA2 (green) (FIG. 6d). Total cells were visualised using DAPI (blue).

### 2.5 Human ES-Cell Derived Neural Precursors are Able to Self Renew for Prolonged Periods In Vitro

Neural precursor cells derived in either differentiation in peptide-supplemented media for 7 days are able to self renewal for extended periods of time (90 days) when cultured in the presence of 8 ng/ml FGF2 as determined by fluorescent flow cytometry analysis of NESTIN expression (FIG. 4b).

### 2.6 Differentiation of ES Cell-Derived Neural Precursor Cells Generate all Three CNS Lineages

To determine if ES cell derived neural precursor cells are able to form mature neuronal cell types in adherent culture we examined a number of glial- and neural-restricted markers. Human ES cells cultured in (a) media alone or (b) peptide-supplemented media (for 7 days) were induced to differentiate towards glial lineages by culture in defined media and plating onto Geltrex-coated plates. Cells were harvested on days 21 and 28 for and assessed for markers of glial cell subsets. Phase contrast images of astrocytes (day 21) from neural precursor cells derived in media alone (FIG. 7ai) and peptide-supplemented media (FIG. 7bi). Positive immunoreactivity of A2B5 (red), an early astrocyte marker at day 21 and GFAP (red) a pan astrocyte marker at day 28 in media only cells (FIGS. 7aii and aiii respectively) and peptide-derived cells (FIGS. 7bii and 7biii). It can be noted that cells derived in peptide-supplemented media display more filamentous localisation of proteins. Phase contrast images of oligodendrocyte cells from neural precursor cells derived in media alone (FIG. 7aiv) and peptide-supplemented media (FIG. 7biv). Positive immunoreactivity of O4 (green) (oligodendrocyte precursor marker) at day 21 was seen extensively in all treatments (FIGS. 7av and bv). Total cells were visualised using DAPI (blue). The differentiated phenotype was further verified by transcript expression analysis of GFAP (astrocyte), Sβ3100 (astrocyte), OLIGO2 (oligodendrocyte) and GAPDH (house-keeping) by RT-PCR on cells cultured for 21 days in glial-differentiation media (FIG. 7c).

Neural differentiation was induced by culture in a defined differentiation medium and plating on orthinine/lamina substrate for 21-28 days. Positive dual immunoreactivity of Neurofilament (red) and βIII-Tubulin (green) at day 21 of neural precursor cells derived in media alone and peptide-supplemented media (FIGS. 8ai and bi respectively) and MAP2 (green) at day 28 (FIGS. 8aii and bii). Total cells were visualised using DAPI (blue). Phase contrast images show presence of different neuron subtypes (day 21-31) from neural precursor cells derived in media alone (FIG. 8aiii-av) and peptide-supplemented media (FIG. 8biii-iv). Although, morphology suggests the presence of motor neurons, TH-positive cells could not be detected following 40 days in culture in any cell lines tested.

### EXPERIMENTAL RESULTS STUDY 2

A further study was undertaken to illustrate the suitability of a range of inhibitors of E-cadherin activity for use in the various aspects of the invention. The inhibitors of E-cadherin activity used in this study, along with two controls (Peptide B and water) were as follows:
1. Peptide A*—SWELYYPLRANL (12-mer that inhibits cell-cell contacts).
2. Peptide B*—SRELYYPLRANL (12-mer with W replaced by R that does not alter cell-cell contacts, but has some cellular effects).
3. Peptide C—SWELYYPL (7-mer that inhibits cell-cell contacts, reduced effect compared to A).
4. E-cadherin neutralising antibody (SHE78.7 clone, available from Invitrogen 13-5700).
5. Experimental vehicle control (water).

### Methods

### Culture of Pluripotent Human iPS Cells

Human iPS cells (hiPSCs) were grown under adherent feeder-free culture conditions. All cells were propagated for a minimum of two passages as feeder-free cultures to exclude unwanted residual mouse fibroblast feeder cells. Cells were cultured in MTesR complete medium (Stem Cell Technologies). Cells were cultured on Matrigel™- (BD Biosciences 356234) coated tissue culture grade plates. Matrigel-treated plates were coated with pre-diluted Matrigel™ (1:100 in DMEM/F12 media) and incubated at room temperature prior to use. Media was replenished every 24 h and cells were passaged upon confluency. Cells were dissociated using trypsin-EDTA.

### E-Cadherin Inhibitor Supplementation During Neural Cell Initiation

Human iPSCs were differentiated as previously described. Peptides A, B or C were supplemented to the media at a final concentration of 1 mM and E-cadherin neutralising antibody supplemented to the media at 2 μg/ml daily for 6-7 days. The equivalent volume of water was added to cultures as a vehicle control.

All other methodology was performed as outlined above in connection with neural differentiation and characterisation of resultant neural progenitor cells (NPCs). The additional supplementation referred to above is employed as pluripotent hiPSCs do not survive well in StemPro medium. Accordingly, supplementation in this manner may be a preferred embodiment of methods of the invention as practiced upon pluripotent stem cells such as iPSCs.

### Results

The results described below are illustrated in FIGS. 9 and 10

The Effect of E-Cadherin on Cell-Cell Contact in Pluripotent hiPSCs

Human iPS cells were grown under standard adherent feeder-free culture conditions prior to induction of differentiation. FIG. 9 shows typical culture morphology of cells grown for 48 h in media supplemented with; (A) peptide A, (B) E-cadherin neutralising antibody, (C) peptide C, (D) peptide B and (E) control. Phase contrast microscope images show that loss of cell-cell contact is achieved in the majority cells (>85%) when cultured with peptide A and E-cadherin neutralising antibody (shown in FIGS. 9A&B respectively), where cells appear largely as single cells compared to the typical compacted ‘colony’ morphology of hiPSCs (shown in FIG. 9E). From these results, it is also evident that cells cultured in the presence of peptide B retain the compacted morphology of hiPSCs, without loss of cell-cell contacts (shown in FIG. 9D). The culture of hiPSCs in the presence of peptide C shows loss of cell-cell contacts in approx. 50-60% of the cell population, however this is markedly lower when compared to peptide A or neutralising antibody.

### Differentiation of Human iPS Cells in Adherent Culture Form Neural Progenitor Cells

To initiate differentiation confluent undifferentiated cells were dissociated and seeded at a low density onto gelatin coated wells in differentiation medium supplemented with/without E-cadherin-inhibitors for 7 days. To identify the phenotype of these cells, cultures were harvested at day 7 and immunofluorescent analysis of the neural progenitor cell marker Nestin was performed (FIG. 10).

(I) Human iPS cells were treated daily for 6-7 days with (A) peptide A, (B) neutralising antibody, (C) peptide C, (D) peptide B and (E) control. Low power magnification (×10) shows distribution of Nestin positive cells (green) with total nuclei stained using DAPI (blue). Nestin positive cells can be observed in all treatments, however cells cultured in the presence of peptide A (A) showed the highest homogenous proportion of Nestin positive cells, this being important in limiting the potential for unwanted cell types during differentiation. Cells treated with the E-cadherin neutralizing antibody (B) also showed a large proportion of Nestin positive cells, as did treatment with peptide C (C). However, cells cultured in the presence of peptide B (does not cause loss of cell-cell contacts) (D) showed the overall lowest proportion of Nestin positive cells (including control cells (E)). (II) Quantification of the number of Nestin positive cells is shown in the chart and was maximal when cells were cultured in media supplemented with peptide A (94%); compared to media supplemented with neutralising antibody (89%), peptide C (76%), peptide B (33%) and control (63%). It must be noted that there were variable results in the number of nestin positive cells when hiPSCs were cultured in the presence of peptide C, with one experiment showing approx. 50% Nestin positive cells only. This variability may be down to failure of abrogation of E-cadherin from the majority of the cell population (see FIG. 1C).

It should also be noted that results achieved using the E-cadherin neutralising antibody show that it also enriches for neural progenitor cells (89% Nestin positive cells), and thus is suitable for use in the methods of the invention. However the relatively lower cost of the non-antibody peptide inhibitor may provide considerable advantages in commercial terms. For example, in conducting the present Study peptide A costs £0.63/ml of media compared to £5.70/ml when using the E-cadherin neutralising antibody.

```
Human E-cadherin sequences
Sequence ID No. 1 DNA sequence - NCBI NM_004360
      1 agtggcgtcg gaactgcaaa gcacctgtga gcttgaggaa gtcagttcag actccagccc
    61 gctccagccc ggcccgaccc gaccgcaccc ggcgcatgcc atcgctcggc gtccccggcc
  121 agccatgggc ccttggagcc gcagcctctc ggcgctgctg ctgctgctgc aggtctcctc
  181 ttggctctgc caggagccgg agccctgcca ccctggcttt gacgccgaga gctacacgtt
  241 caaggtgccc cggcgccacc tggagagagg ccgcgtcctg ggcagagtga attttgaaga
  301 ttgcaccggt cgacaaagga cagcctattt ttccctcgac acccgattca aagtgggcac
  361 agatggtgtg attacagtca aaaggcctct acggtttcat aacccacaga tccatttctt
  421 ggtctacgcc tgggactcca cctacagaaa gttttccacc aaagtcacgc tgaatacagt
  481 ggggcaccac caccgccccc cgccccatca ggcctccgtt tctggaatcc aagcagaatt
  541 gctcacattt cccaactcct ctcctggcct cagaagacag aagagagact gggttattcc
  601 tcccatcagc tgcccagaaa atgaaaaagg cccatttcct aaaaacctgg ttcagatcaa
  661 atccaacaaa gacaaagaag gcaaggtttt ctacagcatc actggccaag gagctgacac
  721 accccctgtt ggtgtcttta ttattgaaag agaaacagga tggctgaagg tgacagagcc
  781 tctggataga gaacgcattg ccacatacac tctcttctct cacgctgtgt catccaacgg
  841 gaatgcagtt gaggatccaa tggagatttt gatcacggta accgatcaga atgacaacaa
  901 gcccgaattc acccaggagg tctttaaggg gtctgtcatg gaaggtgctc ttccaggaac
  961 ctctgtgatg gaggtcacag ccacagacgc ggacgatgat gtgaacacct acaatgccgc
1021 catcgcttac accatcctca gccaagatcc tgagctccct gacaaaaata tgttcaccat
1081 taacaggaac acaggagtca tcagtgtggt caccactggg ctggaccgag agagtttccc
1141 tacgtatacc atggtggttc aagctgctga ccttcaaggt gaggggttaa gcacaacagc
1201 aacagctgtg atcacagtca ctgacaccaa cgataatcct ccgatcttca atcccaccac
1261 gtacaagggt caggtgcctg agaacgaggc taacgtcgta atcaccacac tgaaagtgac
1321 tgatgctgat gcccccaata ccccagcgtg ggaggctgta tacaccatat tgaatgatga
1381 tggtggacaa tttgtcgtca ccacaaatcc agtgaacaac gatggcattt tgaaaacagc
1441 aaagggcttg gattttgagg ccaagcagca gtacattcta cacgtagcag tgacgaatgt
1501 ggtacctttt gaggtctctc tcaccacctc cacagccacc gtcaccgtgg atgtgctgga
1561 tgtgaatgaa gcccccatct ttgtgcctcc tgaaaagaga gtggaagtgt ccgaggactt
1621 tggcgtgggc caggaaatca catcctacac tgcccaggag ccagacacat ttatggaaca
1681 gaaaataaca tatcggattt ggagagacac tgccaactgg ctggagatta atccggacac
1741 tggtgccatt tccactcggg ctgagctgga cagggaggat tttgagcacg tgaagaacag
1801 cacgtacaca gccctaatca tagctacaga caatggttct ccagttgcta ctggaacagg
1861 gacacttctg ctgatcctgt ctgatgtgaa tgacaacgcc cccataccag aacctcgaac
1921 tatattcttc tgtgagagga atccaaagcc tcaggtcata aacatcattg atgcagacct
1981 tcctcccaat acatctccct tcacagcaga actaacacac ggggcgagtg ccaactggac
2041 cattcagtac aacgacccaa cccaagaatc tatcattttg aagccaaaga tggccttaga
2101 ggtgggtgac tacaaaatca atctcaagct catggataac cagaataaag accaagtgac
2161 caccttagag gtcagcgtgt gtgactgtga aggggccgcc ggcgtctgta ggaaggcaca
2221 gcctgtcgaa gcaggattgc aaattcctgc cattctgggg attcttggag gaattcttgc
2281 tttgctaatt ctgattctgc tgctcttgct gtttcttcgg aggagagcgg tggtcaaaga
2341 gcccttactg cccccagagg atgacacccg ggacaacgtt tattactatg atgaagaagg
2401 aggcggagaa gaggaccagg actttgactt gagccagctg cacaggggcc tggacgctcg
2461 gcctgaagtg actcgtaacg acgttgcacc aaccctcatg agtgtccccc ggtatcttcc
2521 ccgccctgcc aatcccgatg aaattggaaa ttttattgat gaaaatctga aagcggctga
2581 tactgacccc acagccccgc cttatgattc tctgctcgtg tttgactatg aaggaagcgg
2641 ttccgaagct gctagtctga gctccctgaa ctcctcagag tcagacaaag accaggacta
2701 tgactacttg aacgaatggg gcaatcgctt caagaagctg gctgacatgt acggaggcgg
2761 cgaggacgac taggggactc gagagaggcg ggccccagac ccatgtgctg ggaaatgcag
2821 aaatcacgtt gctggtggtt tttcagctcc cttcccttga gatgagtttc tggggaaaaa
2881 aaagagactg gttagtgatg cagttagtat agctttatac tctctccact ttatagctct
2941 aataagtttg tgttagaaaa gtttcgactt atttcttaaa gctttttttt ttttcccatc
3001 actctttaca tggtggtgat gtccaaaaga tacccaaatt ttaatattcc agaagaacaa
3061 ctttagcatc agaaggttca cccagcacct tgcagatttt cttaaggaat tttgtctcac
3121 ttttaaaaag aaggggagaa gtcagctact ctagttctgt tgttttgtgt atataatttt
3181 ttaaaaaaaa tttgtgtgct tctgctcatt actacactgg tgtgtccctc tgcctttttt
3241 ttttttttta agacagggtc tcattctatc ggccaggctg gagtgcagtg gtgcaatcac
3301 agctcactgc agccttgtcc tcccaggctc aagctatcct tgcacctcag cctcccaagt
3361 agctgggacc acaggcatgc accactacgc atgactaatt ttttaaatat ttgagacggg
3421 gtctccctgt gttacccagg ctggtctcaa actcctgggc tcaagtgatc ctcccatctt
3481 ggcctcccag agtattggga ttacagacat gagccactgc acctgcccag ctccccaact
3541 ccctgccatt ttttaagaga cagtttcgct ccatcgccca ggcctgggat gcagtgatgt
3601 gatcatagct cactgtaacc tcaaactctg gggctcaagc agttctccca ccagcctcct
3661 ttttattttt ttgtacagat ggggtcttgc tatgttgccc aagctggtct taaactcctg
3721 gcctcaagca atccttctgc cttggccccc caaagtgctg ggattgtggg catgagctgc
3781 tgtgcccagc ctccatgttt taatatcaac tctcactcct gaattcagtt gctttgccca
3841 agataggagt tctctgatgc agaaattatt gggctctttt agggtaagaa gtttgtgtct
3901 ttgtctggcc acatcttgac taggtattgt ctactctgaa gacctttaat ggcttccctc
3961 tttcatctcc tgagtatgta acttgcaatg ggcagctatc cagtgacttg ttctgagtaa
4021 gtgtgttcat taatgtttat ttagctctga agcaagagtg atatactcca ggacttagaa
4081 tagtgcctaa agtgctgcag ccaaagacag agcggaacta tgaaaagtgg gcttggagat
4141 ggcaggagag cttgtcattg agcctggcaa tttagcaaac tgatgctgag gatgattgag
4201 gtgggtatac atcatctctg aaaattctgg aaggaatgga ggagtctcaa catgtgtttc
4261 tgacacaaga tccgtggttt gtactcaaag ccaagaatcc ccaagtgcct gcttttgatg
4321 atgtctacag aaaatgctgg ctgagctgaa cacatttgcc caattccagg tgtgcacaga
4381 aaaccgagaa tattcaaaat tccaaatttt ttcttaggag caagaagaaa atgtggccct
4441 aaagggggtt agttgagggg tagggggtag tgaggatctt gatttggatc tctttttatt
4501 taaatgtgaa tttcaacttt tgacaatcaa agaaaagact tttgttgaaa tagctttact
4561 gtttctcaag tgttttggag aaaaaaatca accctgcaat cactttttgg aattgtcttg
4621 atttttcggc agttcaagct atatcgaata tagttctgtg tagagaatgt cactgtagtt
4681 ttgagtgtat acatgtgtgg gtgctgataa ttgtgtattt tctttggggg tggaaaagga
4741 aaacaattca agctgagaaa agtattctca aagatgcatt tttataaatt ttattaaaca
4801 attttgttaa accataaaaa aaaaaaaa
Sequence ID No. 2 Protein sequence - NCBI AAY68225
          mgpwsrslsa lllllqvssw lcqepepchp gfdaesytft vprrhlergr vlgrvnfedc
    61 tgrqrtayfs ldtrfkvgtd gvitvkrplr fhnpqihflv yawdstyrkf stkvtlntvg
  121 hhhrppphga svsgigaell tfpnsspglr rgkrdwvipp iscpenekgp fpknlvqiks
  181 nkdkegkvfy sitgqgadtp pvgvfiiere tgwlkvtepl dreriatytl fshavssngn
  241 avedpmeili tvtdqndnkp eftqevfkgs vmegalpgts vmevtatdad ddvntynaai
  301 aytilsqdpe lpdknmftin rntgvisvvt tgldresfpt ytlvvqaadl qgeglsttat
  361 avitvtdtnd nppifnptty kgqvpenean vvittlkvtd adapntpawe avytilnddg
  421 gqfvvttnpv nndgilktak gldfeakqqy ilhvavtnvv pfevslttst atvtvdvldv
  481 neapifvppe krvevsedfg vggeitsyta qepdtfmeqk ityriwrdta nwleinpdtg
  541 aistraeldr edfehvknst ytaliiatdn gspvatgtgt lllilsdvnd napipeprti
  601 ffcernpkpq viniidadlp pntspftael thgasanwti qyndptqesi ilkpkmalev
  661 gdykinlklm dnqnkdqvtt levsvcdceg aagverkaqp veaglgipai lgilggilal
  721 lililllllf lrrravvkep llppeddtrd nvyyydeegg geedqdfdls qlhrgldarp
  781 evtrndvapt lmsvprylpr panpdeignf idenlkaadt dptappydsl lvfdyegsgs
  841 eaaslsslns sesdkdqdyd ylnewgnrfk kladmyggge dd
Muse E-cadherin
Sequence ID No. 3 DNA sequence - from NCBI BC098501
          agccgcggcg cactactgag ttcccaagaa cttctgctag actcctgccc ggcctaaccc
    61 ggccctgccc gaccgcaccc gagctcagtg tttgctcggc gtctgccggg tccgccatgg
  121 gagcccggtg ccgcagcttt tccgcgctcc tgctcctgct gcaggtctcc tcatggcttt
  181 gccaggagct ggagcctgag tcctgcagtc ccggcttcag ttccgaggtc tacaccttcc
  241 cggtgccgga gaggcacctg gagagaggcc atgtcctggg cagagtgaga tttgaaggat
  301 gcaccggccg gccaaggaca gccttctttt cggaagactc ccgattcaaa gtggcgacag
  361 acggcaccat cacagtgaag cggcatctaa agctccacaa gctggagacc agtttcctcg
  421 tccgcgcccg ggactccagt catagggagc tgtctaccaa agtgacgctg aagtccatgg
  481 ggcaccacca tcaccggcac caccaccgcg accctgcctc tgaatccaac ccagagctgc
  541 tcatgtttcc cagcgtgtac ccaggtctca gaagacagaa acgagactgg gtcatccctc
  601 ccatcagctg ccccgaaaat gaaaagggtg aattcccaaa gaacctggtt cagatcaaat
  661 ccaacaggga caaagaaaca aaggttttct acagcatcac cggccaagga gctgacaaac
  721 cccccgttgg cgttttcatc attgagaggg agacaggctg gctgaaagtg acacagcctc
  781 tggatagaga agccattgcc aagtacatcc tctattctca tgccgtgtca tcaaatgggg
  841 aagcggtgga ggatcccatg gagatagtga tcacagtgac agatcagaat gacaacaggc
  901 cagagtttac ccaggaggtg tttgagggat ccgttgcaga aggcgctgtt ccaggaacct
  961 ccgtgatgaa ggtctcagcc accgatgcag acgatgacgt caacacctac aacgctgcca
1021 tcgcctacac catcgtcagc caggatcctg agctgcctca caaaaacatg ttcactgtca
1081 atagggacac cggggtcatc agtgtgctca cctctgggct ggaccgagag agttacccta
1141 catacactct ggtggttcag gctgctgacc ttcaaggcga aggcttgagc acaacagcca
1201 aggctgtgat cactgtcaag gatattaatg acaacgctcc tgtcttcaac ccgagcacgt
1261 atcagggtca agtgcctgag aatgaggtca atgcccggat cgccacactc aaagtgaccg
1321 atgatgatgc ccccaacact ccggcgtgga aagctgtgta caccgtagtc aacgatcctg
1381 accagcagtt cgttgtcgtc acagacccca cgaccaatga tggcattttg aaaacagcca
1441 agggcttgga ttttgaggcc aagcagcaat acatccttca tgtgagagtg gagaacgagg
1501 aaccctttga ggggtctctt gtcccttcca cagccactgt cactgtggac gtggtagacg
1561 tgaatgaagc ccccatcttt atgcctgcgg agaggagagt cgaagtgccc gaagactttg
1621 gtgtgggtca ggaaatcaca tcttataccg ctcgagagcc ggacacgttc atggatcaga
1681 agatcacgta tcggatttgg agggacactg ccaactggct ggagattaac ccagagactg
1741 gtgccatttt cacgcgcgct gagatggaca gagaagacgc tgagcatgtg aagaacagca
1801 catatgtagc tctcatcatc gccacagatg atggttcacc cattgccact ggcacgggca
1861 ctcttctcct ggtcctgtta gacgtcaatg ataacgctcc catcccagaa cctcgaaaca
1921 tgcagttctg ccagaggaac ccacagcctc atatcatcac catcttggat ccagaccttc
1981 cccccaacac gtcccccttt actgctgagc taacccatgg ggccagcgtc aactggacca
2041 ttgagtataa tgacgcagct caagaatctc tcattttgca accaagaaag gacttagaga
2101 ttggcgaata caaaatccat ctcaagctcg cggataacca gaacaaagac caggtgacca
2161 cgttggacgt ccatgtgtgt gactgtgaag ggacggtcaa caactgcatg aaggcgggaa
2221 tcgtggcagc aggattgcaa gttcctgcca tcctcggaat ccttggaggg atcctcgccc
2281 tgctgattct gatcctgctg ctcctactgt ttctacggag gagaacggtg gtcaaagagc
2341 ccctgctgcc accagatgat gatacccggg acaatgtgta ttactatgat gaagaaggag
2401 gtggagaaga agaccaggac tttgatttga gccagctgca caggggcctg gatgcccgac
2461 cggaagtgac tcgaaatgat gtggctccca ccctcatgag cgtgccccag tatcgtcccc
2521 gtcctgccaa tcctgatgaa attggaaact tcatcgatga aaacctgaag gcagccgaca
2581 gcgaccccac ggcaccccct tacgactctc tgttggtgtt cgattacgag ggcagtggtt
2641 ctgaagccgc tagcctgagc tcactgaact cctctgagtc ggatcaggac caggactacg
2701 attatctgaa cgagtggggc aaccgattca agaagctggc ggacatgtac ggcggtggcg
2761 aggacgacta ggggactagc aagtctcccc cgtgtggcac catgggagat gcagaataat
2821 tatatcagtg gtctttcagc tccttccctg agtgtgtaga agagagactg atctgagaag
2881 tgtgcagatt gcatagtggt ctcattctcc ttactggact gtctgtgtta ggatggtttt
2941 cactgattgt tgaaatcttt ttttattttt tatttttaca gtgctgagat ataaactgtg
3001 cctttttttg tttgtttgtt tctgtttttg ttcttttgag cagaacaaaa aaaagggacc
3061 actatgcatg ctgcacacgt ctcagattct taggtacaca cctgattctt aggtgcatgc
3121 catagtggga tatgttgctt tgatcagaac ctgcagggag gttttcgggc accacttaag
3181 tttcttggcg ttttcttcaa accgttctct aagatgcatt tttatgaatt ttattaaaga
3241 gttttgttaa aaaaaaaaaa aaaaaaaaaa aaaaaaaaaa aaaaaaaaaa aaaaaaaaaa
3301 aaaaaaaaaa aaaa
Sequence ID No. 4 Protein sequence - from NCBI NP_033994.
mgarcrsfsa lllllqvssw lcqelepesc spgfssevyt fpvperhler ghvlgrvrfe
gctgrprtaf fsedsrfkva tdgtitvkrh lklhkletsf lvrardsshr elstkvtlks
mghhhhrhhh rdpasesnpe llmfpsvypg lrrqkrdwvi ppiscpenek gefpknlvqi
ksnrdketkv fysitgqgad kppvgvfiie retgwlkvtq pldreaiaky ilyshavssn
geavedpmei vitvtdqndn rpeftqpvfe gfvaegavpg tsvmkvsatd adddvntyna
aiaytivsqd pelphknmft vnrdtgvisv ltsgidresy ptytlvvqaa dlqgeglstt
akavitvkdi ndnapvfnps tyqgqvpene vnariatlkv tdddapntpa wkavytvvnd
pdqqfvvvtd pttndgilkt akgldfeakq qyilhvrven eepfegslvp statvtvdvv
dvneapifmp aerrvevped fgvgqeitsy harepdtfmd gkityriwrd tanwleinpe
tgaiftraem dredaehvkn styvaliiat ddgsplatgt gtlllvlldv ndnapipepr
nmqfcgrnpq phiitildpd lppntspfta elthgasvnw tieyndaaqe slilqprkdl
eigeykihlk ladnqnkdqv ttldvhvcdc egtvnncmka givaaglqvp ailgilggil
allilillll lflrrrtvvk epllppdddt rdnvyyydee gggeedqdfd lsqlhrglda
rpevtrndva ptlmsvpgyr prpanpdeig nfidenlkaa dsdptappyd sllvfdyegs
gseaaslssl nssesdqdqd ydylnewgnr fkkladmygg gedd
Sequence ID No. 12 Slug - Human
      1 mprsfivkkh fnaskkpnys eldthtvlls pylyesysmp vlpqpellss gayspitvwt
    61 taapfhaqlp nglsplsgys sslgrvsppp psdtsskdhs gsespisdee erlqsklsdp
  121 haieaekfqc nlcnktystf sglakhkqlh cdaqsrksfs ckycdkeyvs igalkmhirt
  181 htlpcvckic gkafsrpwll qghlrlhtge kpfscphcnr afadrsnlra hlgthsdvkk
  241 yqckncsktf srmsllhkhe esgccvah
Sequence ID No. 13 Slug - Mouse
      1 mprsflvkkh fnsskkpnys eldthtvils pylyesyplp vipkprllts gayspitvwt
    61 ssaaplhspl psglspltgy ssslgrvspp pasdtsdkdh sgsesplsde eerlqpklsd
  121 phaleaekfq cnlcnktyst fsglakhkql hcdaqsrksf sckycdkeyv slgalkmhlr
  181 thtlpcvcki cgkafsrpwl lqghlrthlg ekpfscphcn rafadrsnlr ahlqthsdvk
  241 kyqckncskt fsrmsilhkh eesgccvah
Sequence ID No. 14 Snail - Human
      1 mprsflvrkp sdpnrkpnys elqdsnpeft fqqpydqahl laaipppell nptaslpmli
    61 wdsvlapqaq piawaslrlq esprvaelts lsdedsgkgs qppsppspap ssfsstsvss
  121 leaeayaafp glgqvpkqla qlseakdlqa rkafnckycn keylslgalk mhlrshtlpc
  181 vcgfcgkafs rpwllqghvr thtgekpfsc phcsrafadr snlrahlgth sdvkkyqcqa
  241 cartfsrmsl lhkhqesgcs gcpr
Sequence ID No. 15 Snail - Mouse
      1 mprsflvrkp sdprrkpnys elqdacveft fqqpydqahl laaipppevl npaaslptll
    61 wfsllvpqvr pvawallplr espkavelts lsdedsgkss qppsppspap ssfsstsass
  121 leaeafiafp glgqfpkqla rlsvakdpqs rkifnckycn keylslgalk mhlrshtlpc
  181 vcitcgkafs rpwltqghvr thtgekpfsc shcnrafadr snlrahlqth sdvkryqcqa
  241 cartfsrmsl thkhqesgcs ggpr
Sequence ID No. 16 SIP1 - Human
      1 mkqplmadgp rckrckqanp rrknvvnydn vvdtgsetde edklhlaedd glanpldqet
    61 spasvpnhes sphvsgallp reeeedeire ggvehpwhnn ellqasvdgp eemkedydtm
  121 gpeatiqtai nngtvknanc tsdfeeyfak rkleerdgha vsleeylqrs dtailypeap
  181 eelsrlgtpe angqeendlp pgtpdafaql ltcpycdrgy krltslkehl kyrhekneen
  241 fscplcsytf ayrtqlerhm vthkpgtdqh qmltqgagnr kfkctecgka fkykhhlkeh
  301 lrihsgekpy acpnckkrfs hsgsysshls skkclgllsv ngrmrnnlkt gsspnsvsss
  361 ptnsaltqlr nklengkpls mseqtgllki ktepldfndy kvlmaihgfs gtspfmnggl
  421 gatsplgvhp saqspmqhlg vgmeapllgf plmnsnlsev qkvlqlvdnt vsrqkmdcka
  481 eelsklkgyh mkdpcsqpee qgvtspnlpp vglpvvshng atksiidytl ekvneakacl
  541 qslttdsrrq lsnlkkekir tlldlvtddk mlenhnistp fscqfckesf pgplplhqhe
  601 rylckmneel kavkqpheni vpnkagvfvd nkalllssvl sekgmtspin pykdhmsvlk
  661 ayyamnmepn sdellkisla vglpqefvke wfeqrkvyqy snsrspsler sskplapnsn
  721 pptkdsllpr spvkpmdsit spslaelhns vtncdpplrl tkpshftnik pvekldhsrs
  781 ntpsplnlss tssknshsss ytpnsfssee lqaepldlsl pkqmkepksi iatknktkas
  841 slsldhnsvs sssensdepl nliflkkefs nsnnldnkst npvfsmnpfs akplytalpp
  901 qsafppatfm ppvqtsipgl rpypgldqms flphmaytyp tgaatfadmq qrrkyqrkqg
  961 fqgelldgaq dymsglddmt dsdscisrkk lkktesgmya cdlcdktfqk sssllrhkye
1021 htgkrphqcq ickkafkhkh hliehsrlhs gekpyqcdkc gkrfshsgsy sqhmnhrysy
1081 ckreaeerea aerearekgh leptellmnr aylqsitpqg ysdseeresm prdgesekeh
1141 ekegedgygk lgrqdgdeef eeeeeesenk smdtdpetir deeetgdhsm ddssedgkme
1201 tksdheednm edgm
Sequence ID No. 17 SIP1 - Mouse
      1 mkqplmadgp rckrrkqanp rrknvvnydn vvdagsetde edkihlaedd stanpldqdt
    61 spasmpnhes sphmsqgllp reeeeeelre svvehswhsg ellqasvagp eemkedydam
  121 gpeatiqtti nngtvknanc tsdfeeyfak rklesrdgha vsieeylqrs dtallypeap
  181 eelsrlgtpe angqeendlp pgtpdafaql ltcpycdrgy krltslkehl kyrhekneen
  241 fscplcsytf ayrtqlerhm vthkpgtdqh qmltqgagnr kfkctecgka fkykhhlkeh
  301 lrihsgekpy ecpnckkrfs hsgsysshls skkcigllsv ngrmrnnikt gsspnsvsss
  361 ptnsaltqlr nklengkpls mseqtgllki ktepldfndy kvlmathgfs gsspfmnggl
  421 gatsplgvhp saqspmqhlg vgmeapllgf ptmnsnlsev qkvlqlvdnt vsrqkmdckt
  481 edisklkgyh mkdpcsgpee ggvlspnipp vglpvvshng atksiidytl ekvneakacl
  541 qslttdsrrq isnlkkeklr tlldlvtddk mienhsistp fscqfckesf pgpfplhqhe
  601 rylckmneei kavlqphenl vpnkagvfvd nkalllssvl sekgltspln pykdhmsvlk
  661 ayyamnmepn sdellkisia vglpqefvke wfeqrkvyqy snsrspsler tskplapnsn
  721 pttkdsllpr spvkpmdslt spslaelhns vtscdpplrl tksshftnlk avdkldhsrs
  781 ntpsplnlss tssknshsss ytpnsfssee lqaepldlsl pkqmrepkgi iatknklkat
  841 slntdhnsvs sssensdepl nltlikkefs nsnnldnksn npvfgmnpfs akplytplpp
  901 qsafppatim ppvqlslpgl rpypgldqms flphmaytyp tgaatfadmq grrkyqrkqg
  961 fqgdlldgaq dymsglddmt dsdsclsrkk ikktesgmya cdlcdktfqk sssllrhkye
1021 htgkrphqcq ickkafkhkh hliehsrlhs gekpyqcdkc gkrfshsgsy sqhmnhrysy
1081 ckreaeerea aerearekgh lgptellmnr aylqsltpqg ysdseeresm prdgesekeh
1141 ekegeegygk lrrrdgdeee eeeeeesenk smdtdpetir deeetgdhsm ddssedgkme
1201 tksdheednm edgmg
Sequence ID No. 18 E2A - Human - Amino acid sequence
MNQPQRMAPVGTDKELSDLLDFSMMFPLPVTNGKGRPASLAGAQ
FGGSGLEDRPSSGSWGSGDQSSSSFDPSRTFSEGTHFTESHSSLSSSTFLGPGLGGKS
GERGAYASFGRDAGVGGLTQAGFLSGELALNSPGPLSPSGMKGTSQYYPSYSGSSRRR
AADGSLDTQPKKVRKVPPGLPSSVYPPSSGEDYGRDATAYPSAKTPSSTYPAPFYVAD
GSLHPSAELWSPPGQAGFGPMLGGGSSPLPLPPGSGPVGSSGSSSTFGGLHQHERMGY
QLHGAEVNGGLPSASSFSSAPGATYGGVSSHTPPVSGADSLLGSRGTTAGSSGDALGK
ALASIYSPDHSSNNFSSSPSTPVGSPQGLAGTSQWPRAGAPGALSPSYDGGLHGLQSK
IEDHLDEAIHVLRSHAVGTAGDMHTLLPGHGALASGFTGPMSLGGRHAGLVGGSHPED
GLAGSTSLMHNHAALPSQPGTLPDLSRPPDSYSGLGRAGATAAASEIKREEKEDEENT
SAADHSEEEKKELKAPRARTSPDEDEDDLLPPEQKAEREKERRVANNARERLRVRDIN
EAFKELGRMCQLHLNSEKPQTKLLILHQAVSVILNLEQQVRERNLNPKAACLKRREEE
KVSGVVGDPQMVLSAPHPGLSEAHNPAGHM
Sequence ID No. 19 E2A - human - DNA encoding sequence
      1 gcctgaggtg cccgccctgg ccccaggaga atgaaccagc cgcagaggat ggcgcctgtg
    61 ggcacagaca aggagctcag tgacctcctg gacttcagca tgatgttccc gctgcctgtc
  121 accaacggga agggccggcc cgcctccctg gccggggcgc agttcggagg ttcaggtctt
  181 gaggaccggc ccagctcagg ctcctggggc agcggcgacc agagcagctc ctcctttgac
  241 cccagccgga ccttcagcga gggcacccac ttcactgagt cgcacagcag cctctcttca
  301 tccacattcc tgggaccggg actcggaggc aagagcggtg agcggggcgc ctatgcctcc
  361 ttcgggagag acgcaggcgt aggcggcctg actcaggctg gcttcctgtc aggcgagctg
  421 gccctcaaca gccccgggcc cctgtcccct tcgggcatga aggggacctc ccagtactac
  481 ccctcctact ccggcagctc ccggcggaga gcggcagacg gcagcctaga cacgcagccc
  541 aagaaggtcc ggaaggtccc gccgggtctt ccatcctcgg tgtacccacc cagctcaggt
  601 gaggactacg gcagggatgc caccgcctac ccgtccgcca agacccccag cagcacctat
  661 cccgccccct tctacgtggc agatggcagc ctgcacccct cagccgagct ctggagtccc
  721 ccgggccagg cgggcttcgg gcccatgctg ggtgggggct catccccgct gcccctcccg
  781 cccggtagcg gcccggtggg cagcagtgga agcagcagca cgtttggtgg cctgcaccag
  841 cacgagcgta tgggctacca gctgcatgga gcagaggtga acggtgggct cccatctgca
  901 tcctccttct cctcagcccc cggagccacg tacggcggcg tctccagcca cacgcggcct
  961 gtcagcgggg ccgacagcct cctgggctcc cgagggacca cagctggcag ctccggggat
1021 gccctcggca aagcactggc ctcgatctac tccccggatc actcaagcaa taacttctcg
1081 tccagccctt ctacccccgt gggctccccc cagggcctgg caggaacgtc acagtggcct
1141 cgagcaggag cccccggtgc cttatcgccc agctacgacg ggggtctcca cggcctgcag
1201 agtaagatag aagaccacct ggacgaggcc atccacgtgc tccgcagcca cgccgtgggc
1261 acagccggcg acatgcacac gctgctgcct ggccacgggg cgctggcctc aggtttcacc
1321 ggccccatgt cgctgggtgg gcggcacgca ggcctggttg gaggcagcca ccccgaggac
1381 ggcctcgcag gcagcaccag cctcatgcac aaccacgcgg ccctccccag ccagccaggc
1441 accctccctg acctgtctcg gcctcccgac tcctacagtg ggctagggcg agcaggtgcc
1501 acggcggccg ccagcgagat caagcgggag gagaaggagg acgaggagaa cacgtcagcg
1561 gctgaccact cggaggagga gaagaaggag ctgaaggccc cccgggcccg gaccagccca
1621 gacgaggacg aggacgacct tctcccccca gagcagaagg ccgagcggga gaaggagcgc
1681 cgggtggcca ataacgcccg ggagcggctg cgggtccgtg acatcaacga ggcctttaag
1741 gagctggggc gcatgtgcca actgcacctc aacagcgaga agccccagac caaactgctc
1801 atcctgcacc aggctgtctc ggtcatcctg aacttggagc agcaagtgcg agagcggaac
1861 ctgaatccca aagcagcctg tttgaaacgg cgagaagagg aaaaggtgtc aggtgtggtt
1921 ggagaccccc agatggtgct ttcagctccc cacctaggcc tgagcgaagc ccacaacccc
1981 gccgggcaca tgtgaaaggt atgcctccgt gggacgagcc acccgctttc agccctgtgc
2041 tctggcccca gaagccggac tcgagacccc gggcttcatc cacatccaca cctcacacac
2101 ctgttgtcag catcgagcca acaccaacct gacaaggttc ggagtgatgg gggcggccaa
2161 ggtgagactg ggtccaggag ctccctgggg ccctggccta ccactcactg gcctcgctcc
2221 ccctgtcccc gaatctcagc caccgtgtca ctctgtgacc tgtcccatgg atcctgaaac
2281 tgcatcttgg ccctgttgcc tgggctgaca ggagcatttt ttttttttcc agtaaacaaa
2341 acctgaaagc aagcaacaaa acatacactt tgtcagagaa gaaaaaaatg ccttaactat
2401 aaaaagcgga gaaatggaaa catatcactc aagggggatg ctgtggaaac ctggcttatt
2461 cttctaaagc caccagcaaa ttgtgcctaa gcgaaatatt ttttttaagg aaaataaaaa
2521 cattagttac aagatttttt ttttcttaag gtagatgaaa attagcaagg atgctgcctt
2581 tggtctctgg tttttttaag ctttttttgc atatgttttg taaggaacaa atttttttgt
2641 ataaaagtcc cgtgtctctc gctatttctg ctgctgttcc tagactgagc attgcatttc
2701 ttgatcaacc agatgattaa acgttgtatt aaaaagaccc cgtgtaaacc tgagcccccc
2761 ccgtcccccc ccccggaagc cactgcacac agacagacgg ggacaggcgg cgggtctttt
2821 gtttttttga tgttgggggt tctcttggtt ttgtcatgtg gaaagtgatg cgtgggcgtt
2881 ccctgatgaa ggcaccttgg ggcttccctg ccgcatcctc tcccctcagg aaggggactg
2941 acctgggctt gggggaaggg acgtcagcaa ggtggctctg accctcccag gtgactctgc
3001 caagcagctg tggccccagc ggtaccctac acaacgccct ccccaggccc ccctaagctg
3061 ctctcccttg gaacctgcac agctctctga aatggggcat tttgttggga ccagtgaccc
3121 ctggcatggg gaccacaccc tggagcccgg tgctggggac ctcctggaca ccctgtcctt
3181 cactccttgc cccagggacc caggctcatg ctctgaactc tggctgagag gagtctgctc
3241 aggagccagc acaggacacc ccccacccca ccccaccatg tccccattac accagagggc
3301 catcgtgacg tagacaggat gccaggggcc tgaccagcct ccccaatgct ggggagcatc
3361 cctggcctgg ggccacacct gctgccctcc ctctgtgtgg tccaagggca agagtggctg
3421 gagccggggg actgtgctgg tctgagcccc acgaaggcct tgggctgtgg ctccgaccct
3481 gctgcagaac cagcagggtg tcccctcggg cccatctgtg tcccatgtcc cagcacccag
3541 gcctctctcc aggtctcctt ttctggtctt ttgccatgag ggtaaccagc tcttcccagc
3601 tggctgggac tgtcttgggt ttaaaactgc aagtctccta ccctgggatc ccatccagtt
3661 ccacacgaac tagggcagtg gtcactgtgg cacccaggtg tgggcctggc tagctggggg
3721 ccttcatgtg cccttcatgc ccctccctgc attgaggcct tgtggacccc tgggctggct
3781 gtgttcatcc ccgctgcagg tcgggcgtct ccccccgtgc cactcctgag actccaccgt
3841 tacccccagg agatcctgga ctgcctgact cccctcccca gactggcttg ggagcctggg
3901 ccccatggta gatgcaaggg aaacctcaag gccagctcaa tgcctggtat ctgcccccag
3961 tccaggccag gcggagggga ggggctgtcc ggctgcctct cccttctcgg tggcttcccc
4021 tgcgccctgg gagtttgatc tcttaaggga acttgcctct ccctcttgtt ttgctcctgc
4081 cctgccccta ggtctgggtg gcagtggccc catagcctct ggaactgtgc gttctgcata
4141 gaattcaaac gagattcacc cagcgcgagg aggaagaaac agcagttcct gggaaccaca
4201 attatggggg gtggggggtg tgatctgagt gcctcaagat ggttttcaaa aaattttttt
4261 taaagaaaat aattgtatac gtgtcaacac agctggctgg atgattggga ctttaaaacg
4321 accctctttc aggtggattc agagacctgt cctgtatata acagcactgt agcaataaac
4381 gtgacatttt ataaag
Sequence ID No. 20 E2A - Mouse - Amino acid sequence
MMNQSQRMAPVGSDKELSDLLDFSMMFPLPVANGKSRPASLGGT
QFAGSGLEDRPSSGSWGSSDQNSSSFDPSRTYSEGAHFSDSHSSLPPSTFLGAGLGGK
GSERNAYATFGRDTSVGTLSQAGFLPGELSLSSPGPLSPSGIKSSSQYYPSFPSNPRR
RAADGGLDTQPKKVRKVPPGLPSSVYPPSSGDSYSRDAAAYPSAKTPSSAYPSPFYVA
DGSLHPSAELWSTPSQVGFGPMLGDGSSPLPLAPGSSSVGSGTFGGLQQQDRMGYQLH
GSEVNGSLPAVSSFSAAPGTYSGTSGHTPPVSGAAAESLLGTRGTTASSSGDALGKAL
ASIYSPDHSSNNFSPSPSTPVGSPQGLPGTSQWPRAGAPSALSPNYDAGLHGLSKMED
RLDEAIHVLRSHAVGTASDLHGLLPGHGALTTSFTGPMSLGGRHAGLVGGSHPEEGLT
SGASLLHNHASLPSQPSSLPDLSQRPPDSYSGLGRAGTTAGASEIKREEKEDEEIASV
ADAEEDKKDLKVPRTRTSSTDEVLSLEEKDLRDRERRMANNARERVRVRDINEAFREL
GRMCQLHLKSDKAQTKLLILQQAVQVILGLEQQVRERNLNPKAACLKRREEEKVSGVV
GDPQLPLSAAHPGLGEAHNPAGHL
Sequence ID No. 21 E2A - Mouse - DNA encoding sequence
      1 gcgccggcgg ctgcgggcgt agcgggccac cgcgggccac cgccgcgcgc cgccgcctct
    61 gctacagtcc cttcccgcgg ggcctgctct gagagaagct cgagagagac caggcgacgc
  121 gaacgcgagt ggggaggagg aaggacgcgc gacccogagc cctgcgcgct cccgccgccc
  181 acgcgcgacc ctcggggacg cgcccgccac ccttttgtcc ccggggtccc cgagggcggt
  241 gggcagcagg gagccccggt gcacccggtg catgcccccg cccagcaggg ctgtctctag
  301 acctggggga cgcaccccag ttccaacacc tgctgtcctg ggtggatgat gaaccagtct
  361 cagagaatgg cacccgtggg ctctgacaag gaactgagtg acctcctgga cttcagcatg
  421 atgttcccgc tacctgtggc caataggaag agccggcccg cctccctcgg gggaacccag
  481 tttgcaggct caggactgga ggaccgaccc agctcaggct cctggggcag cagtgaccag
  541 aacagttctt cctttgaccc tagccggaca tacagcgaag gtgcccactt cagtgactcc
  601 cacagcagcc tgccgccttc cacgttccta ggagctgggc ttggaggcaa gggcagtgag
  661 cggaatgcct atgccacctt tgggagagac accagtgttg gcaccttgag tcaggctggc
  721 ttcctgccag gtgagctgag cctcagcagt cccgggccac tgtccccatc gggcatcaag
  781 agcagctccc agtattaccc ctcattcccc agcaaccctc gtcggagagc tgcagatggt
  841 ggcctggata ctcagccgaa gaaggtccgg aaggttccgc ctggtctccc ttcctcggtg
  901 tatccgccca gctcaggtga cagctacagc agggatgctg cagcctaccc ctccgccaag
  961 acccccagca gcgcttaccc ctccccottc tacgtggcag atggcagcct gcacccatca
1021 gctgagctct ggagtacgcc tagccaggtg ggctttgggc ccatgctagg tgacggctct
1081 tcccctctgc cccttgcacc gggcagcagc tccgtgggca gtggtacctt tgggggcctc
1141 cagcagcagg atcgcatggg ctaggagctg catggatctg aggttaatgg ctagctccca
1201 gctgtatcca gcttttcggc tgcccctggc acttacagtg ggacttccgg ccacacgccc
1261 cctgtgagtg gggccgcagc tgaaagcctc ctaggcaccc gagggactac agccagcagc
1321 tcaggggatg cccttgggaa ggcactggcc tcgatctact ccccggatca ctccagcaat
1381 aatttctcac ctagcccctc aacgcctgtg ggttcacccc agggcctgcc agggacatca
1441 cagtggcccc gggcaggagc gcccagtgcc ttatccccca actacgatgc aggtctccat
1501 ggcctgagca agatggagga ccgcttggac gaggccatcc atgtcctgcg aagccacgct
1561 gttggcaccg ctagcgatct ccatgggctt ttgcctggcc atggcgcact gaccacgagc
1621 ttcaccggcc ccatgtcact gggcgggcgg catgccggcc tggtcggggg aagccatcct
1681 gaggagggcc tcacaagtgg ggccagtctt ttgcataacc atgccagcct ccccagccag
1741 cccagttccc tccctgacct ctcacagaga cctcccgact cctatagtgg actcgggagg
1801 gcaggcacaa cagcgggtgc cagcgagatc aagcgggagg agaaagagga tgaggaaatc
1861 gcatcagtag ccgacgccga agaggacaag aaggacctga aggtcccacg cacgcgcacc
1921 agcagtacag atgaggtgct gtccctggag gagaaggacc tgagggaccg ggagaggcgt
1981 atggccaata acgctcggga gcgggtgcgc gtgcgggaca ttaacgaggc cttccgggag
2041 ctgggccgca tgtgccagct gcacctcaag tcggataagg cgcagaccaa gctgctcatc
2101 ctgcagcagg cggtgcaggt catcctgggc ctggagcagc aggtgcgaga acgcaacctg
2161 aaccccaaag cagcctgctt gaagcggagg gaggaggaga aggtgtctgg cgtggtcggg
2221 gacccacagc tgcccctgtc agccgcccac ccgggcctgg gtgaggccca caacccagcc
2281 gggcacctgt gagccgtcac agcttcttcg ttggaccagg gaccaccata tctctgcccg
2341 gggtgcatca ggacggttct ggatgagaca ggtctbcatc gaagcatgag cagagagagg
2401 gctctgggga cacttcaggg cctggggagg gtggcactga acagctccct gcttggcccc
2461 agtgaccaag cagaaaagtt ccttcctctc ggttaaccag aactggaaac aaagcagcat
2521 gctccctttt caaaaaggaa agaaagatgc cttaactatg taagacggaa gagtcggacc
2581 gtgccctggc agggcggcct gggactggct tctacttcag agccaccagc acatcgtgcc
2641 taagcatttt tcgttttttt aaaggagaat aaaggaacat tagttttcag attttttttt
2701 taaatgtaga caaaagttag caagaacgag gccttccgtg tctttttttt ttcccttagc
2761 ttttttttcc gtatgttttg taaggaacaa atttttgtat aaaagtctca tgtctgtttc
2821 tgtttctaga aaaaaaaaaa aaaaaaaaaa aaaaaatatt taaaaaaaaa aaaaaaaaaa
2881 aaaaaaaaaa aaaaaaaa
5 x human E-cadherin in pRNAtin-H1.2neo
DQ090940 Homo sapiens cadherin 1, type 1, E-cadherin (epithelial)
(CDH1)gene, complete cds.
Sequence ID No. 22. siRNA insert 1: 76 bp. start at 2258
BamH I Hind III
```

| | |
|---|---|
| GGATCCCGTATTTACGACCTTTCTTGGCATTGATATCCGTGCCAAGAAAGGTCGTAAATATTTTTTCCAAAAGCTT | 2258-2278 |

\hat{ }| Antisense | Loop | Sense | Termination Signal
Sequence ID No. 23. siRNA insert 2: 76 bp. start at 339
BamH I Hind III

| | |
|---|---|
| GGATCCCGTTTCTTGAGCCATAAATGCTCTTGATATCCGGAGCATTTATGGCTCAAGAAATTTTTTCCAAAAGCTT | 339-359 |

\hat{ }| Antisense | Loop | Sense | Termination Signal
Sequence ID No. 24. siRNA insert 3: 76 bp. start at 478
BamH I Hind III

| | |
|---|---|
| GGATCCCGTTAGTGAGTCAGCAAATTGATTTGATATCCGATCAATTTGCTGACTCACTAATTTTTTCCAAAAGCTT | 478-498 |

\hat{ }| Antisense | Loop | Sense | Termination Signal
Sequence ID No. 25. siRNA insert 4: 76 bp. start at 986
BamH I Hind III

| | |
|---|---|
| GGATCCCGTGTGAGCCATGAGCCACTGAGTTGATATCCGCTCAGTGGCTCATGGCTCACATTTTTTCCAAAAGCTT | 986-1006 |

\hat{ }| Antisense | Loop | Sense | Termination Signal
Sequence ID No. 26. siRNA insert 5: 76 bp. start at 2976
BamH I Hind III

| | |
|---|---|
| GGATCCCGCATAGTCAACAACCAGGCAGGTTGATATCCGCCTGCCTGGTTGTTGACTATGTTTTTTCCAAAAGCTT | 2976-2996 |

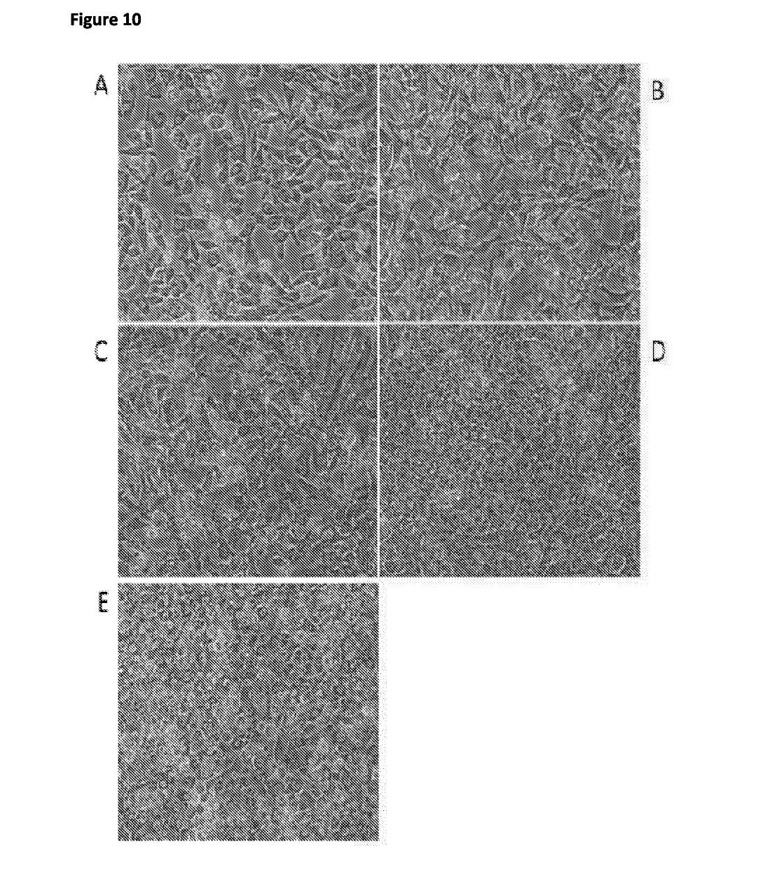

```
              \hat{ }| Antisense | Loop | Sense | Termination Signal
FIG. 14 - part 2
3 x mouse E-cadherin in pRNAtin-H1.2neo
BC098501 Mus musculus cadherin 1, mRNA (cDNA clone MGC: 107495
IMAGE: 30023851), complete cds.
Sequence ID No. 27. siRNA insert 1: 76 bp. start at 2126
BamH I Hind III
GGATCCCGTTGTTCTGGTTATCCGCGAGCTTGATATCCGGCTCGCGGATAACCAGAACAATTTTTTCCAAAAGCTT
              \hat{ }| Antisense | Loop | Sense | Termination Signal
Sequence ID No. 28. siRNA insert 2: 76 bp. start at 1385
BamH I Hind III
GGATCCCGTCTGTGACGACAACGAACTGCTTGATATCCGGCAGTTCGTTGTCGTCACAGATTTTTTCCAAAAGCTT
              \hat{ }| Antisense | Loop | Sense | Termination Signal
Sequence ID No. 29. siRNA insert 3: 76 bp. start at 369
BamH I Hind III
GGATCCCGTAGATGCCGCTTCACTGTGATTTGATATCCGATCACAGTGAAGCGGCATCTATTTTTTCCAAAAGCTT
              \hat{ }| Antisense | Loop | Sense | Termination Signal
```

1. A method of producing neural precursor cells, the method comprising:
providing an inhibitor of E-cadherin activity to a population of the cells having neural potential;
inducing cell stress among the population of cells; and
culturing the surviving cells until neural precursor cells are produced.

2. A method of adapting a cell in vitro for therapeutic use, the method comprising:
providing an inhibitor of E-cadherin activity to a population of cells having neural potential;
inducing cell stress among the population of cells;
culturing the surviving cells until neural precursor cells are produced;
culturing the neural precursor cells until neural cells are produced; and
formulating the neural precursor cells or neural cells in a composition suitable for administration to a patient.

3. The method according to claim 1, wherein the cells having neural potential are stem cells selected from the group consisting of: embryonic stem cells; cord blood stem cells; mesenchymal stem cells; induced pluripotent stem cells (iPSCs) and a human embryonic stem cell line.

4. The method according to claim 3, wherein the stem cells are selected from the group consisting of: multipotent cells; totipotent cells; and pluripotent cells.

5-6. (canceled)

7. The method according to claim 1, wherein the inhibitor of E-cadherin activity is an exogenous inhibitor of E-cadherin activity, wherein the exogenous inhibitor of E-cadherin activity is provided in a cell culture medium.

8. (canceled)

9. The method according to claim 1, wherein the inhibitor of E-cadherin activity is selected from the group consisting of the peptide SWELYYPLRANL (SEQ ID NO. 1) and the peptide SWELYYPL (SEQ ID NO. 26).

10. The method according to claim 1, wherein the inhibitor of E-cadherin activity comprises the peptide SWELYYPLRANL (SEQ ID NO. 1), or the peptide SWELYYPL (SEQ ID NO. 26).

11. The method according to claim 1, wherein the inhibitor of E-cadherin activity is selected from the group consisting of: E-cadherin neutralising antibodies; inhibitors of the E-cadherin HAV domain; inhibitors of tryptophan 2 binding sites; E-cadherin neutralising aptamers; RNAi molecules that inhibit E-cadherin; Slug; Snail; SIP1; E2A; peptides comprising Trp156; and peptides comprising the amino acid sequence CHAVC (SEQ ID NO. 3).

12. The method according to claim 1, wherein the inhibitor of E-cadherin activity is an endogenous inhibitor of E-cadherin activity.

13. The method according to claim 11, wherein the inhibitor of E-cadherin activity is selected from the group consisting of: E-cadherin neutralising antibodies; inhibitors of the E-cadherin HAV domain; inhibitors of tryptophan 2 binding sites; and peptides comprising the amino acid sequence CHAVC (SEQ ID NO. 3).

14. The method according to claim 11, wherein the E-cadherin neutralising antibody is SHE78.7.

15. The method according to claim 1, wherein the inhibitor of E-cadherin activity is provided to the cells prior to the induction of cell stress, or concurrently with the induction of cell stress.

16. (canceled)

17. The method according to any preceeding claim 1, wherein the means of inducing physiological stress are selected from the group consisting of: withdrawal of an agent that is beneficial to cultured cells; withdrawal of serum from the medium provided to the cell population; increasing the temperature to which the population of cells is exposed; increasing or decreasing pH of the medium in which the population of cells is grown; and providing a cytotoxic agent to the population of cells.

18-19. (canceled)

20. The method according to claim 1, wherein the neural precursor cells produced express nestin.

21-27. (canceled)

28. A kit comprising:
an inhibitor of E-cadherin activity;
a serum-free cell medium; and
serum or a serum-replacement composition.

29. The kit according to claim 28, wherein the inhibitor of E-cadherin activity is selected from the group consisting of the peptide SWELYYPLRANL (SEQ ID NO. 1), the peptide SWELYYPL (SEQ ID NO. 26), E-cadherin neutralising antibodies, inhibitors of the E-cadherin HAV domain, inhibitors of tryptophan 2 binding sites, E-cadherin neutralising aptamers, RNAi molecules that inhibit E-cadherin, Slug, Snail, SIP1, E2A, peptides comprising Trp156, peptides comprising the amino acid sequence CHAVC (SEQ ID NO. 3), and E-cadherin neutralising antibody SHE78.7.

30. A cell culture medium comprising an inhibitor of E-cadherin activity at a concentration of between approximately 450 μM and approximately 1.1 mM.

31. cell culture medium according to claim 30, wherein the inhibitor of E-cadherin activity comprises the peptide SWELYYPLRANL (SEQ ID NO. 1).

32. The method according to claim 1, wherein the neural precursor cells are further cultured to produce at least one of the following cells: neural cells, glial cells or neuronal cells.

33. The cells produced by the method of claim 32.
